(19) United States
(12) Patent Application Publication
(10) Pub. No.: US 2022/0233652 A1
(43) Pub. Date: Jul. 28, 2022

(54) Pharmaceutical Compositions Comprising Integration-Promoting Peptides

(71) Applicant: Zion Medical B.V., Rehovot (IL)

(72) Inventors: Esmira Naftali, Petach Tikva (IL); Zyon Ayni, Rehovot (IL)

(21) Appl. No.: 17/270,262

(22) PCT Filed: Aug. 22, 2019
(86) PCT No.: PCT/IL2019/050944

§ 371 Date: Feb. 22, 2021

Related U.S. Application Data
(60) Provisional application No. 62/721,630, filed on Aug. 23, 2018.
(60) Provisional application No. 62/744,143, filed on Oct. 11, 2018.

(51) Int. Cl. A61K 38/45; C12N 9/12; A61K 47/26; A61P 31/18; A61P 35/00; A61K 31/513; A61K 31/426; C12N 15/86; A61K 39/395

(52) U.S. Cl. CPC A61K 38/45; C12Y 207/07049; C12N 9/1276; A61K 47/26; A61P 31/18; C12N 2740/15042; A61K 31/513; A61K 31/426; C12N 15/86; A61K 39/3955; A61P 35/00

(57) ABSTRACT

The present invention provides pharmaceutical compositions of an integration-promoting peptide and hydroxyl halide salts of said peptide. The pharmaceutical compositions of the present invention are stable and maintain the peptide soluble upon administration. Methods of treating viral infection and cancer with the peptide salts and compositions thereof are also provided.

### FIELD OF THE INVENTION

The present invention relates to stable pharmaceutical compositions of integration-promoting peptides and to halide salts of said peptides.

### BACKGROUND OF THE INVENTION

It was previously suggested that by cooperation between retrovirus-derived integrase enzymes, accessory DNA and integration promoting agents, the integrase enzymes are capable of translocating into the nuclei of target cells, where they induce double-stranded breaks (DSBs) in the chromosomal DNA. It was hypothesized that the accumulation of DSBs exhausts the natural DNA repair capabilities of the cell, and drive the cell to apoptosis. WO 2010/041241 describes several integration-promoting peptides derived from the HIV-1 integrase protein, and their effect on HIV-infected cells.

WO 2018/215999 further exemplified use of such integration-promoting peptides in methods for inducing DNA breaks in specifically-targeted cells, in particular cancer and HIV-infected cells, thereby promoting cell death.

Peptide compositions are inherently unstable due to sensitivity towards chemical and physical degradation. Chemical degradation involves change of covalent bonds, oxidation, hydrolysis, racemization or crosslinking. Physical degradation involves conformational changes relative to the native structure of the peptide, i.e. secondary and tertiary structure, such as aggregation, precipitation or adsorption to surfaces. In addition, many peptides, particularly hydrophobic ones, have low solubility in physiological conditions. There is a need for development of pharmaceutical compositions and formulations which allow high stability and bioavailability and convenient administration of such peptides.

### SUMMARY OF THE INVENTION

The present invention is directed to stable pharmaceutical composition for parenteral administration of specific peptides, in particular peptides comprising the amino acid sequence TAVQMAVFIHNFKRK (SEQ ID NO: 2) derived from the HIV-1 protein integrase. These peptides were previously shown as being insoluble in physiological pH and osmolarity conditions. The compositions of the present invention comprise, according to some embodiments, from 0.1 to 30 mg/ml of the peptide or a salt thereof, and are in conformity with requirements from parenteral compositions, i.e. the composition is isotonic and has a physiologically acceptable pH between 4 to 9. Specifically, intravenous and subcutaneous compositions are provided as well as solid compositions. Also provided are halide salts of the peptides used in the compositions.

According to one aspect, the present invention provides a pharmaceutical composition comprising from about 0.1 to about 30 mg/ml of a salt of a peptide of 15 to 30 amino acids that comprises the sequence TAVQMAVFIHNFKRK (SEQ ID NO: 2), or of a derivative, fragment or analog thereof, from about 0.3 wt % to about 15 wt % of a poloxamer having the structure of Formula I,

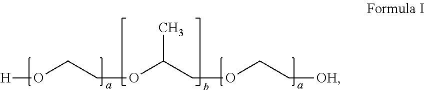

wherein a is an integer from 50 to 120, and b is an integer from 15 to 40 and a pharmaceutically acceptable carrier, wherein the pH of the composition is between 4 to about 7.5.

According to some embodiments, the peptide comprises the amino acid sequence WTAVQMAVFIHNFKRK (SEQ ID NO: 1), or a derivative or analog thereof.

According to some embodiments, the peptide consists of the amino acid sequence WTAVQMAVFIHNFKRK (SEQ ID NO: 1, also denoted hereinafter “INS”).

According to some embodiments, the peptide contained in the pharmaceutical compositions is in the form of halide salt. According to specific embodiments, the salt of the peptide is a hydrochloride salt. According to specific embodiments, the salt of the INS is a hydrochloride salt (INS HCl).

According to some embodiments, the composition comprises from about 1 mg/ml to about 20 mg/ml of INS HCl.

According to another embodiment, a in Formula I is an integer from 60 to 90 and b is an integer from 25 to 35. In some particular embodiments, a=80 and b=27 (poloxamer 188). According to some embodiments, the pharmaceutical composition comprises from about 0.1 wt % to about 10 wt % of poloxamer 188. According to some embodiments, the composition comprises from about 1 to about 10 wt % of poloxamer 188 and from about 5 mg/ml to about 15 mg/ml of the peptide INS HCl. According to other embodiments, the pharmaceutical composition comprises from about 0.5 wt % to about 5 wt % of poloxamer 188 and/or from about 1 mg/ml to about 10 mg/ml of the peptide INS HCl.

A pharmaceutical composition according to some embodiments further comprises a saccharide. According to some embodiments, the saccharide is selected from a disaccharide and polysaccharide. According to some embodiment, the composition comprises from about 1 wt % to about 20 wt % of the saccharide. According to other embodiments, the composition comprises from about 5 wt % to about 12 wt % of the saccharide. According to some embodiments, the saccharide is a disaccharide. According to a specific embodiment, the disaccharide is lactose.

According to some embodiments, the present invention provides a pharmaceutical composition comprising from about 0.5 to about 10 wt % of poloxamer 188, from about 5 mg/ml to about 15 mg/ml of HCl salt of a peptide consisting of the amino acid sequence WTAVQMAVFIHNFKRK (SEQ ID NO: 1), and from about 5 to about 12 wt % of lactose.

According to other embodiments, the pharmaceutical composition comprises from about 8 to 12 wt % lactose, from about 0.5 to about 2 wt % of poloxamer 188, and from about 1 mg/ml to about 10 mg/ml of INS HCl. According to further embodiments, the pharmaceutical composition comprises from about 8 to 12 wt % lactose, from about 0.8 to about 2 wt % of poloxamer 188, and from about 5 mg/ml to about 10 mg/ml of INS HCl. In yet other embodiments, the pharmaceutical composition comprises from about 8 to 12 wt % lactose, from about 0.8 to about 2 wt % of poloxamer 188, and from about 1 mg/ml to about 15 mg/ml of INS HCl.

According to some embodiments, the pharmaceutical composition further comprises lentivirus particles.

According to some embodiments, the pharmaceutical composition of the present invention further comprises a targeting moiety, e.g. a moiety that directs the active ingredients to a specific population of cells.

According to some embodiments, the composition is a liquid or semi-liquid composition. According to another embodiment, the composition is a solution. According to specific embodiments the solution is aqueous solution. According to yet another embodiment, the composition is a stable composition.

According to one embodiment, the composition is a parenteral composition, i.e. a composition suitable for parenteral administration such as intravenous, intramuscular and subcutaneous administration.

According to some embodiments, the composition further comprising an additional active agent. According to some embodiments, the active agent is an anti-viral agent. According to some specific embodiments, the anti-viral agent is a protease inhibitor. According to yet other embodiments, the additional active agent is an anti-cancer agent.

According to some embodiment, the pharmaceutical composition is a solid composition. According to some embodiments, the solid composition is a lyophilized composition. According to some embodiments, the solid composition is in the form of powder.

According to another aspect, the present invention provides a stable and soluble pharmaceutical composition comprising the peptide INS (SEQ ID NO: 1), for use in treating a disease, wherein treating comprises destroying a specific population of target cells. According to some embodiments, the composition is for use in treating subjects positive for HIV-1 and for treatment of acquired immune deficiency syndrome (AIDS). According to some embodiments, the composition is for use in treating cancer.

According to some embodiments, the composition for use of the present invention is administered parenterally. According to some embodiments, the composition for use is administered via a route selected from the group consisting of: intramuscular (IM), intravenous (IV) and subcutaneous (SC). According to yet other embodiments, the composition is administered directly to the diseased or injured site, for example intratumorally.

According to some embodiments, the composition is administered in combination with: (i) a linear molecule of double-stranded DNA (dsDNA) comprising long term repeat (LTR) sequences recognized by the integrating enzyme of (ii), and (ii) an integrating enzyme, capable of entering the nuclei of cells after binding to the LTR sequences of the dsDNA molecule of (i) and creating multiple double strand breaks (DSBs) in the chromosomal DNA of the cells. According to some embodiments, the linear molecule of dsDNA comprising LTR sequences recognized by the integrating enzyme is part of a lentivirus particle.

According to some embodiments, the composition is administered in combination with a targeting moiety. According to some embodiments the targeting moiety is a molecule capable of binding to a cell receptor or marker. According to some embodiments the targeting moiety is an antibody capable of binding to a tumor antigen. According to a specific embodiment, the targeting moiety is an antibody against CD24.

According to some embodiments, the composition is administered together with lentiviruses or lentivirus particles.

According to some embodiment, the composition is administered in combination with (i) a lentivirus particle comprising a linear molecule of double-stranded DNA (dsDNA) comprising long term repeat (LTR) sequences recognized by the integrating enzyme of (ii), (ii) an integrase, capable of entering the nuclei of cells after binding to the LTR sequences of the dsDNA molecule of (i) and creating multiple double strand breaks (DSBs) in the chromosomal DNA of the cells, and (iii) an antibody capable of binding CD24 expressed in cancer cells.

According to a further aspect, the present invention provides a solid pharmaceutical composition comprising a salt of a peptide consisting of the amino acid sequence of SEQ ID NO: 1, and a poloxamer having a structure of Formula I,

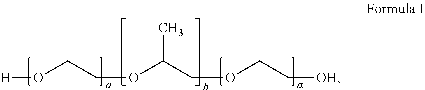

wherein a is an integer from 50 to 120, and b is an integer from 15 to 40 and wherein the weight ratio between said salt and said poloxamer is from about 10:5 to about 1:1500. According to some embodiments, the salt of said peptide is hydrochloride salt (INS HCl) and the poloxamer is poloxamer 188. According to other embodiments, the weight ratio between said salt and said poloxamer 188 is from about 10:1 to about 1:1000. According to some embodiments, the weight ratio in said solid composition between said salt and said poloxamer 188 is from about 2:1 to about 1:20 or from about 1:1 to about 1:5. According to some embodiments, the solid pharmaceutical composition further comprises a saccharide selected from a disaccharide and polysaccharide. In some embodiments, the disaccharide is lactose. According to one embodiment, the weight ratio between said salt and lactose is from about 4:1 to about 1:2000. According to some embodiments, the weight ratio between said salt and poloxamer 188 is between about 3:1 to 1:20 and the weight ratio between said salt and lactose is between about 4:1 to about 1:24. According to some embodiments, the solid pharmaceutical composition is a lyophilized composition.

According to one embodiment, the solid pharmaceutical composition of the present invention upon reconstitution provides a stable parenteral pharmaceutical composition according to the present invention. Thus, according to some embodiments, the present invention provides reconstituted pharmaceutical compositions suitable for parenteral administration. According to one embodiment, such a reconstituted pharmaceutical composition is for use in treating a disease selected from cancer, HIV-1 infection and AIDS.

According to another aspect, the present invention provides a hydrogen halide salt of a peptide comprising the amino acid sequence of SEQ ID NO: 2, wherein said peptide consists of from 15 to 30 amino acids. According to some embodiments, the peptide comprises the amino acid sequence of SEQ ID NO: 1. According to some embodiments, the peptide consists of an amino acid sequence selected from SEQ ID NO: 1 and SEQ ID NO: 2. According to some embodiments, the hydrogen halide salt is a hydrochloride salt. In some embodiments, the present invention provides a hydrochloride salt of the peptide denoted INS consisting of the amino acid sequence set forth in SEQ ID NO: 1.

The present invention also provides methods of treating a disease comprising administering to a subject in need thereof a stable pharmaceutical composition comprising therapeutically effective amount of the peptide INS, or of a salt thereof. According to some embodiment, the subject is a human subject.

According to some embodiments, treatment results in destroying a specific population of target cells. According to some embodiments the specific population of target cells comprises cells selected from HIV-infected cells and tumor cells.

According to some embodiments, the composition is administered parenterally. According to some embodiment, the treated disease is selected from viral infection and cancer. According to some embodiments, the viral infection is HIV-1 infection. According to a specific embodiment, the treated disease is AIDS caused by HIV-1 infection. According to some embodiments, the method of treatment is part of a treatment regimen comprising administration of at least one protease inhibitor. According to some embodiments, the treatment method is part of a regimen comprising administration of a cocktail of at least two anti-HIV-1 agents.

According to some embodiments, a method of treatment an HIV-1 infection is provided comprising administering to a subject in need thereof a pharmaceutical composition comprising a salt of a peptide consisting of the amino acid sequence of SEQ ID NO: 1, and a poloxamer having a structure of Formula I,

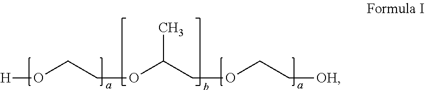

wherein a is an integer from 50 to 120, and b is an integer from 15 to 40 and wherein the weight ratio between said salt and said poloxamer is from about 10:5 to about 1:1500, in combination with treatment with at least one additional anti-HIV-1 agent.

According to some embodiments, a method of treatment an HIV-1 infection is provided comprising administering to a subject in need thereof a pharmaceutical composition comprising 0.5 to about 10 wt % of poloxamer 188, from about 5 mg/ml to about 15 mg/ml of HCl salt of a peptide consisting of the amino acid sequence WTAVQMAVFIHNFKRK (SEQ ID NO: 1), and from about 5 to about 12 wt % of lactose.

According to some embodiments, the method of treating HIV-1 infection further comprising administering to said subject at least one protease inhibitor. According some specific embodiments, the method comprises administering of the protease inhibitors lopinavir and ritonavir.

According to some embodiment, a method of treating cancer is provided comprising administering to a subject in need thereof, a pharmaceutical composition comprising a salt of a peptide consisting of the amino acid sequence of SEQ ID NO: 1, and a poloxamer having a structure of Formula I,

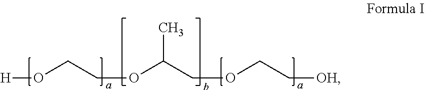

wherein a is an integer from 50 to 120, and b is an integer from 15 to 40 and wherein the weight ratio between said salt and said poloxamer is from about 10:5 to about 1:1500.

According to a specific embodiment, the method of treatment cancer comprises administering to a subject in need thereof a pharmaceutical composition comprising 0.5 to about 10 wt % of poloxamer 188, from about 5 mg/ml to about 15 mg/ml of HCl salt of a peptide consisting of the amino acid sequence WTAVQMAVFIHNFKRK (SEQ ID NO: 1), and from about 5 to about 12 wt % of lactose.

According to some embodiments, the method of treating cancer comprises co-administration of (i) a linear molecule of double-stranded DNA (dsDNA) comprising long term repeat (LTR) sequences recognized by the integrating enzyme of (ii), and (ii) an integrating enzyme, capable of entering the nuclei of cells after binding to the LTR sequences of the dsDNA molecule of (i) and creating multiple double strand breaks (DSBs) in the chromosomal DNA of the cells. According to some embodiments, the linear molecule of dsDNA comprising LTR sequences recognized by the integrating enzyme is part of a lentivirus particle.

According to some embodiments, the method of treating cancer comprises administering a composition according to the present invention in combination with a targeting moiety. According to some embodiments the targeting moiety is a molecule capable of binding to a cell receptor or marker. According to some embodiments the targeting moiety is an antibody capable of binding to a tumor antigen. According to a specific embodiment, the targeting moiety is an antibody against CD24.

According to some embodiments, the method of treating cancer comprises administering a pharmaceutical composition comprising from about 8 to 12 wt % lactose, from about 0.8 to about 2 wt % of poloxamer 188, and from about 1 mg/ml to about 15 mg/ml of the peptide INS HCl, in combination with (i) a lentivirus particle comprising a linear molecule of double-stranded DNA (dsDNA) comprising long term repeat (LTR) sequences recognized by the integrating enzyme of (ii), (ii) an integrase, capable of entering the nuclei of cells after binding to the LTR sequences of the dsDNA molecule of (i) and creating multiple double strand breaks (DSBs) in the chromosomal DNA of the cells, and (iii) an antibody capable of binding a tumor antigen expressed in cancer cells. According to some embodiments, the antibody is directed to human CD24.

According to some embodiments the cancer is a solid cancer. According to yet other embodiments, the cancer is selected from the group consisting of: sarcoma, lung cancer and pancreatic cancer. According to other embodiments, the cancer treatable with the compositions of the present invention is characterized by over expression of CD24. According to specific embodiments, the cancer characterized by over expression of CD24 is selected from the group consisting of: ovarian cancer, breast cancer, prostate cancer, bladder cancer, renal cancer, pancreatic cancer, lung cancer, sarcoma and nonsmall cell carcinoma.

### BRIEF DESCRIPTION OF DRAWINGS

FIG. 1 shows HPLC chromatograms of the peptide INS (SEQ ID NO: 1) in water.

FIG. 2 shows images of selected formulations comprising 5 mg/ml of the peptide INS (SEQ ID NO: 1) F4, F7 and F10 (numbers 1, 3, and 5 in the figure, respectively) and their dilution in PBS (numbers 2, 4, and 6 in the figure, respectively).

FIG. 3 shows HPLC chromatograms of the Formulation F6 at t=0 and at t=24 h.

FIG. 4 shows HPLC chromatograms of Formulation F4 maintained at room temperature or at 4° at t=0 and at t=24 h.

FIG. 5 shows HPLC chromatograms of Formulation F7 maintained at room temperature or at 4° at t=0 and at t=24 h.

FIG. 6 shows HPLC chromatograms of Formulation F10 maintained at room temperature or at 4° C. at t=0 and at t=24 h.

FIG. 7 shows HPLC chromatograms of Formulation F43 (5 mg/ml of INS) maintained at different conditions at t=0 (7), t=24 h (6: 5° C.; 5: 25° C.) and after 1 week (2: 25° C.; 3: 5° C.) or lyophilized (1: −20° C. 7 days; 4: −20° C. 1 day); 8—INS standard 0.5 mg/ml.

FIG. 8 shows HPLC chromatograms of Formulation F44 maintained at different conditions at t=0(7), t=24 h (6: 5° C.; 5: 25° C.) and after 1 week (2: 25° C.; 3: 5° C.) or lyophilized (1: −20° C. 7 days; 4: −20° C. 1 day); 8—INS standard 0.5 mg/ml.

FIG. 9 shows dynamic light scattering (DLS) image of Formulation 43 at initial state; mean density 1.4 nm.

FIG. 10 shows DLS image of Formulation 43 maintained at 4° C. for 1 week; mean density 12 nm.

FIG. 11 shows DLS image of Formulation 43 maintained at 25° C. for 1 week; mean density 14.9 nm.

FIG. 12 shows DLS image of Formulation 43 maintained at −20° C. for 1 week; mean density 13.4 nm.

FIG. 13 shows DLS image of Formulation 44 at initial state; mean density 2.8 nm.

FIG. 14 shows DLS image of Formulation 44 maintained at 4° C. for 1 week; mean density 14.5 nm.

FIG. 15 shows DLS image of Formulation 44 maintained at 25° C. for 1 week; mean density 10.8 nm.

FIG. 16 shows DLS image of Formulation 44 maintained at −20° C. for 1 week; mean density 13.4 nm.

FIGS. 17A and 17B show images of Lyophilized formulation F43 (FIG. 17A) and its reconstituted solution (FIG. 17B).

FIG. 18 shows HPLC chromatograms of lyophilized-reconstituted formulation at t=0 (5-filtered 0.22 μm; and 6—non-unfiltered)—t=24 h (3—filtered 0.22 μm; and 4—non-unfiltered), and t=7 days (1—filtered 0.22 μm; and 2—non-unfiltered); 7—INS standard; 8—blank: 9.5 wt % lactose+0.8 wt % poloxamer P188.

FIG. 19 shows the efficacy of INS treatment, together with lentiviruses, in lung cancer model.

FIG. 20 shows the efficacy of INS treatment, together with lentiviruses, in pancreatic cancer model at day 17.

FIGS. 21A and 21B show representative examples from two different HIV patients, of viral (HIV-1) load in human subjects after 5 weeks of twice weekly SC administration of INS, 0.05-0.2 mg/kg for the patient presented in FIG. 21A and 0.2-0.4 mg/kg for the patient presented by FIG. 21B, in a formulation comprising 1% poloxamer 188 and 9% lactose anhydrous formulation at pH ˜5.

FIGS. 22A and 22B show representative examples from two different HIV patients (001/A012 and 007/A028), of viral (HIV-1) load in two human subjects after 5 weeks of SC administration of 0.2 or 0.4 mg/kg INS, in a formulation comprising 1% poloxamer 188 and 9% lactose anhydrous formulation at pH ˜5, twice a week, in combination with two antiviral drugs, lopinavir 800 mg plus ritonavir 200 mg daily.

FIG. 23 shows a representative example of CD4 T-cell count in an HIV patient (009/A056) treated only with INS (0.2-0.4 mg/kg) for 5 weeks and additional 5 weeks with INS (0.4 mg/kg), in combination with lopinavir 800 and ritonavir as described above.

### DETAILED DESCRIPTION OF THE INVENTION

According to one aspect, the present invention provides a pharmaceutical composition comprising from about 0.1 to about 30 mg/ml of a salt of a peptide comprising the amino acid sequence set forth in SEQ ID NO: 2 (TAVQMAVFIHNFKRK), from about 0.3 wt % to about 15 wt % of a poloxamer having the structure of Formula 0,

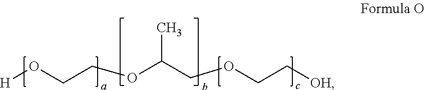

and a pharmaceutically acceptable carrier, wherein the pH of the composition is between 4 to about 7.5 and wherein a and c are each independently an integer from 50 to 120, and b is an integer from 15 to 40, and the peptide consists of 15 to 40 amino acids.

The term “poloxamer” as used herein refer to non-ionic poly (ethylene oxide) (PEO)-poly (propylene oxide) (PPO) copolymers.

According to one embodiment, the peptide comprises the amino acid sequence set forth in SEQ ID NO: 1 (WTAVQMAVFIHNFKRK). According to some embodiments, the peptide consists of 10 to 30 amino acids. According to one embodiment, the peptide consists of the amino acid sequence WTAVQMAVFIHNFKRK (SEQ ID NO: 1), denoted “INS”. Thus, the term “peptide consisting of amino acid sequence SEQ ID NO: 1”, “peptide consisting of amino acid sequence WTAVQMAVFIHNFKRK” and “INS” are used herein interchangeably. According to another embodiment, the peptide consists of the amino acid sequence set forth in SEQ ID NO: 2 (TAVQMAVFIHNFKRK).

According to any one of the above embodiments, any water-soluble salt of a peptide of the invention is contemplated. According to one embodiment, the salt is trifluoroacetate salt. According to another embodiment, the salt is acetate salt. According to one embodiment, the salt is hydroxyl halide salt. According to one embodiment, the hydroxyl halide salt is selected from HCl, HBr and HI salt.

According to one embodiment, a and c in Formula 0, are each independently an integer from 55 to 115, from 60 to 110, from 65 to 105, from 70 to 100 or from 75 to 90. According to another embodiment, b is an integer from 15 to 35, or from 20 to 30. According to some embodiment, a=c. According to such embodiment, the poloxamer has a structure of Formula I:

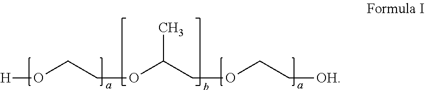

According to one embodiment, the present invention provides a pharmaceutical composition comprising from about 0.1 to about 30 mg/ml of a salt of a peptide consisting of 15 to 30 amino acid and comprising the amino acid sequence set forth in SEQ ID NO: 1 (WTAVQMAVFIHNFKRK), from about 0.3 wt % to about 15 wt % of a poloxamer having the structure of Formula I, and a pharmaceutically acceptable carrier, wherein the pH of the composition is between 4 to about 7.5 and

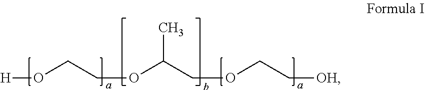

wherein a is an integer from 50 to 120, and b is an integer from 15 to 40.

According to one embodiment, the present invention provides a pharmaceutical composition comprising from about 0.1 to about 30 mg/ml of a salt of a peptide consisting of the amino acid sequence set forth in SEQ ID NO: 1, from about 0.3 wt % to about 15 wt % of a poloxamer having the structure of Formula I, and a pharmaceutically acceptable carrier, wherein the pH of the composition is between 4 to about 7.5 and

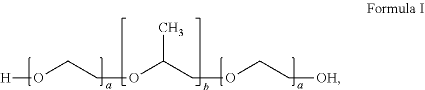

wherein a is an integer from 50 to 120, and b is an integer from 15 to 40.

The term “pharmaceutical composition” as used herein refers to a composition comprising at least one active agent as disclosed herein optionally formulated together with one or more pharmaceutically acceptable carriers. Formulation of the pharmaceutical composition may be adjusted according to their intended use and administration route. In particular, the pharmaceutical composition may be formulated using a method known in the art so as to provide rapid, continuous or delayed release of the active ingredient after administration to mammals. According to one embodiment, the pharmaceutical composition is formulated for a parenteral administration.

Thus in some embodiments, the present invention provides a parenteral pharmaceutical composition comprising from about 0.1 to about 30 mg/ml of a salt of a peptide consisting of amino acid sequence SEQ ID NO: 1, from about 0.3 wt % to about 15 wt % of a poloxamer having the structure of Formula I,

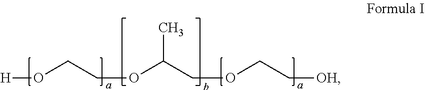

and a pharmaceutically acceptable carrier, wherein the pH of the composition is between 4 to about 7.5 and wherein a is an integer from 50 to 120, and b is an integer from 15 to 40, and the pharmaceutical composition is configured for parenteral administration.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art.

Pharmaceutical compositions adapted for parenteral administration include, but are not limited to, aqueous and non-aqueous sterile injectable solutions or suspensions, which can contain antioxidants, buffers, bacteriostats and solutes that render the compositions substantially isotonic with the blood of an intended recipient. Such compounds do not decrease the stability or the solubility of the peptide.

According to one embodiment, a is an integer from 55 to 115, from 60 to 110, from 65 to 105, from 70 to 100 or from 75 to 90. According to another embodiment, b is an integer from 15 to 35, or from 20 to 30. According to some embodiments, a is an integer from 60 to 90 and b is an integer from 25 to 35. According to one embodiment, a=80 and b=27, and such a poloxamer is referred hereinafter as poloxamer 188. According to some embodiments, poloxamer 188 has a molecular weight between 7600 to 9600. According to one embodiment, poloxamer 188 has a molecular weight of 7800 to 9400, 8000 to 9200 or 8400 to 8800 Dalton.

According to one embodiment, the composition comprises from 0.1% to 5 wt % poloxamer. According to another embodiment, the composition comprises from 5 wt % to 10 wt % poloxamer. According to a further embodiment, the composition comprises from 10 wt % to 15 wt % poloxamer. According to certain embodiments, the composition comprises from 15 wt % to 20 wt % poloxamer. According to one embodiment, the composition comprises from 0.5 wt % to 12 wt %, from 0.6 wt % to 10 wt %, from 0.8 wt % to 8 wt %, from 1 wt % to 6 wt %, from 2 wt % to 5 wt %, from 3 wt % to 4 wt % of poloxamer. According to one embodiment, the pharmaceutical composition comprises from 0.5 wt % to 2 wt %, from 0.6 wt % to 1.8 wt % from 0.7 wt % to 1.6 wt % from 0.8 wt % to 1.4 wt % or from 0.9 wt % to 1.2 wt % of the poloxamer. According to another embodiment, the pharmaceutical composition comprises from 0.6 wt % to 1.2 wt % of the poloxamer. According to one embodiment, poloxamer is poloxamer 188.

According to some embodiment, the composition comprises from 0.1 to 30 mg/ml of INS (SEQ ID NO: 1) salt. According to one embodiment, the composition comprises from 0.5 mg/ml to 25 mg/ml, from 1 mg/ml to 20 mg/ml, from 2.5 mg/ml to 15 mg/ml, from 5 mg/ml to 12 mg/ml, from 6 mg/ml to 10 mg/ml or from 7 mg/ml to 8 mg/ml of INS salt. According to another embodiment, the pharmaceutical composition comprises from 0.5 to 12 wt % from 0.7 to 10 wt % or from 1 to 10 wt % of INS as a salt. According to some embodiment, the pharmaceutical composition comprises 1, 2, 2.5, 3, 4, 5, 6, 7, 7.5, 8, 9, or 10 wt % of the peptide consisting of SEQ ID NO: 1. According to any one of the above embodiments, the peptide of the present invention is a salt. According to one embodiment, the peptide consists of amino acid sequence SEQ ID NO: 1. According to one embodiment, the composition comprises a trifluoroacetate salt of INS. According to another embodiment, the composition comprises an acetate salt of INS. According to one embodiment, the composition comprises a hydroxyl halide salt of INS. According to one embodiment, the hydroxyl halide salt is selected from HCl, HBr and HI salt. According to one embodiment, the composition comprises an HCl salt of INS, denoted also as “INS HCl”.

According to the teaching of the present invention, the concentration or the amount when refers to INS salt relates to the peptide component of the salt. Thus, the statement that the composition comprises 10 mg/ml of INS HCl means that the composition comprises 10 mg/ml of the peptide consisting of SEQ ID NO:1. To calculate the content of the whole INS salt, the number should be multiplied by a correction factor of about 1.1, depending on the salt.

According to one embodiment, the composition comprises from 0.5 mg/ml to 25 mg/ml, from 1 mg/ml to 20 mg/ml, from 2.5 mg/ml to 15 mg/ml, from 5 mg/ml to 12 mg/ml from 6 mg/ml to 10 mg/ml or from 7 mg/ml to 8 mg/ml of INS peptide as HCl salt. According to one embodiment, the pharmaceutical composition comprises from 0.5 to 12 wt % from 0.7 to 10 wt % or from 1 to 10 wt % of the peptide set forth in SEQ ID NO: 1 as HCl salt. According to some embodiment, the pharmaceutical composition comprises 1, 2, 2.5, 3, 4, 5, 6, 7, 7.5, 8, 9, or 10 wt % of the peptide set forth in SEQ ID NO: 1 as HCl salt.

According to one embodiment, the composition comprises from 0.6 to 10 wt % of poloxamer 188 and from 1 mg/ml to 20 mg/ml, from 2.5 mg/ml to 15 mg/ml, from 1 to 10 mg/ml, from 2 to 10 mg/ml or from 5 mg/ml to 10 mg/ml of the peptide INS HCl. According to another embodiment, the composition comprises from 0.8 to 8 wt % poloxamer 188 and from 1 mg/ml to 20 mg/ml, from 2.5 mg/ml to 15 mg/ml or from 5 mg/ml to 10 mg/ml of the peptide INS HCl. According to a further embodiment, the composition comprises from 1 to 5 wt % poloxamer 188 and from 1 mg/ml to 20 mg/ml, from 2.5 mg/ml to 15 mg/ml, from 1 to 10 mg/ml, from 2 to 10 mg/ml or from 5 mg/ml to 10 mg/ml of the peptide INS HCl. According to yet another embodiment, the composition comprises from 0.8 to 2 wt % poloxamer 188 and from 1 mg/ml to 20 mg/ml, from 2.5 mg/ml to 15 mg/ml, from 1 to 10 mg/ml, from 2 to 10 mg/ml or from 5 mg/ml to 10 mg/ml of INS HCl.

According to one embodiment, the composition comprises from 0.3 wt % to 5 wt % of poloxamer 188 and from 1 mg/ml to 10 mg/ml of the peptide INS HCl. According to another embodiment, the composition comprises from 0.5 wt % to 2 wt % poloxamer 188 and from 1 to 5 mg/ml, from 1 to 5 or from 2 mg/ml to 4 mg/ml of the peptide INS HCl. According to any one of the above embodiments, the pharmaceutical composition has a pH of about 4 to 7, about 4.5 to about 6.5 or from about 5 to about 6.

According to any one of the above embodiments, the pharmaceutical composition further comprises a saccharide. According to one embodiment, the saccharide is selected from a disaccharide or polysaccharide. According to one embodiment, the saccharide is a disaccharide. According to one embodiment, the disaccharide is selected from lactose, sucrose, lactulose, maltose, trehalose, cellobiose, and chitobiose. According to one embodiment, the disaccharide is lactose.

According to another embodiment, the saccharide is a polysaccharide. According to one embodiment, the polysaccharide is dextran.

According to some embodiments, the pharmaceutical composition comprises from about 1 wt % to about 20 wt % of the saccharide. According to one embodiment, the composition comprises from 2 wt % to 18 wt %, from 4 wt % to 16 wt %, from 6 wt % to 14 wt %, from 8 to 12 wt % of the saccharide. According to one embodiment, the saccharide is a disaccharide.

According to one embodiment, the composition comprises from 1 wt % to 20 wt %, from 2 wt % to 18 wt %, from 4 wt % to 16 wt %, from 6 wt % to 14 wt % from 8 to 12 wt % of lactose. According to one embodiment, the composition comprises from 6 to 12 wt % of lactose. According to some embodiment, the lactose is anhydrous.

According to some embodiments, the pharmaceutical composition is devoid of a compound selected from Tween and ethanol.

According to one embodiment, the pharmaceutical composition comprises from about 0.5 to about 10 wt % of poloxamer 188, from about 1 mg/ml to about 20 mg/ml of HCl salt of the peptide consisting of amino acid sequence WTAVQMAVFIHNFKRK (SEQ ID NO: 1) and from about 5 to about 12 wt % of lactose.

According to another embodiment, the pharmaceutical composition of the present invention comprises from about 0.8 to about 2 wt % of poloxamer 188, from about 1 mg/ml to about 15 mg/ml or to about 10 mg/ml of the peptide INS HCl and from about 8 to about 12 wt % of lactose.

According to any one of the above embodiments, the pH of the composition is from 4.5 to 6.

According to yet another embodiment, the pharmaceutical composition comprises from about 0.8 to about 2 wt % of poloxamer 188, from about 10 mg/ml to about 20 mg/ml of INS HCl and from about 8 to about 12 wt % of lactose. According to a further embodiment, the pharmaceutical composition comprises from about 8 to 12 wt % lactose, from about 0.5 to about 2 wt % of poloxamer 188, and from about 1 mg/ml to about 5 mg/ml of the peptide INS HCl. According to one embodiment, the pharmaceutical composition comprises from about 8 to 12 wt % lactose, from about 0.8 to about 2 wt % of poloxamer 188, from about 5 mg/ml to about 10 mg/ml of the peptide INS HCl. According to another embodiment, the pharmaceutical composition comprises from about 8 to 12 wt % lactose, from about 0.8 to about 2 wt % of poloxamer 188, and from about 1 mg/ml to about 15 mg/ml of the peptide INS HCl. According to yet another embodiment, the pharmaceutical composition comprises from about 8 to 12 wt % lactose, from about 0.5 to about 2 wt % of poloxamer 188, and from about 1 mg/ml to about 12 mg/ml of the peptide INS HCl. According to some embodiments, the pH of the composition is from 4.5 to 6.

According to one embodiment, the pharmaceutical composition of the present invention comprises from about 0.5 to about 10 wt % of poloxamer 188, from about 5 mg/ml to about 15 mg/ml of HCl salt of the peptide consisting of amino acid sequence SEQ ID NO: 1 and from about 5 to about 12 wt % of lactose. According to one embodiment, the composition comprises from 2.5 to 20 mg/ml of the peptide INS HCl, 1% Poloxamer 188 and 9% Lactose anhydrous, and the composition has pH of about 5. According to another embodiment, the composition comprises from 2.5 to 20 mg/ml of the peptide INS HCl, 0.8% Poloxamer 188 and 9.5% Lactose anhydrous, and the composition has pH of about 5. According to some such embodiments, the composition comprises 2.5, 5, 10, 15 or 20 mg/ml of the peptide INS HCl. According to other embodiments, the pH is from about 4.5 to about 5.5

According to some embodiment, the pharmaceutical composition further comprises at least one buffer. The terms “buffer,” “buffering system,” and/or “buffer solution” refer to compounds which reduce the change of pH upon addition of small amounts of acid or base, or upon dilution. The term “buffering agent” refers to a weak acid or weak base in a buffer solution. According to one embodiment, the buffer is an acetate buffer.

According to any one of the above embodiments, the composition is a liquid composition. According to another embodiment, the composition is a semi-liquid composition. According to some embodiments the liquid or semi-liquid composition is an aqueous composition.

According to any one of the above embodiments, the composition is a solution. The term “solution” as used herein refers to a clear, homogeneous liquid dosage form that contains one or more chemical substances dissolved in a solvent or in a mixture of mutually miscible solvents.

According to one embodiment, the composition is a clear solution. As used herein, the term “clear solution” refers to essentially transparent solutions devoid of particles above 100 nm. Alternatively, the term “clear solution” refers to essentially transparent solutions devoid of particles above 50 nm. Alternatively, the term “clear solution” refers to essentially transparent solutions devoid of particles above 40 nm.

The terms “substantially devoid”, “essentially devoid”, “devoid”, “does not include” and “does not comprise” may be used interchangeably and refer to composition that does not include, contain or comprise a particular compound or particles, e.g. said composition comprises less than 0.1%, less than 0.01%, or less than 0.001% of the such compounds or particles. In some embodiments, the term devoid contemplate composition comprising traces of the devoid compounds or particles.

According to any one of the above embodiments, the composition is a stable composition.

The term “stable” as used herein refer to a composition, for example a solution, in which at least 80% of the peptide remains dissolved for at least 24 hours at room temperature (about 25° C.). According to one embodiment, at least 80 wt % of the peptide maintains dissolved for at least 2 days, at least 5 days, at least 7 days or at least 14 days. According to another embodiment, at least 85 wt % of the peptide remains dissolved for at least 2 days, at least 5 days, at least 7 days or at least 14 days. According to a certain embodiment, at least 90 wt % or 95 wt % of the peptide remains dissolved for at least 2 days, at least 5 days, at least 7 days or at least 14 days.

According to one embodiment, the composition is stable upon dilution with a phosphate buffered saline (PBS). According to one embodiment, the composition remains clear solution upon dilution with PBS. According to another embodiment, the composition remains a stable for at least 1, 2, 3, 5, 7 10 or 14 days upon dilution in PBS. According to one embodiment, the dilution is 2, 5 or 10 times dilution. According to one embodiment, no more that 10%, 5% or 3% of the INS peptide precipitates upon dilution of the pharmaceutical composition with PBS. According to one embodiment, dilution is 10 times dilution.

According to any one of the above embodiments, the pharmaceutical composition of the present invention is a parenteral composition, i.e. the composition is formulated for parenteral administration. According to one embodiment, parenteral administration is selected from intravenous, intramuscular and subcutaneous administration. Thus, according to one embodiment, the pharmaceutical composition of the present invention is formulated for an administration route selected from intravenous, intramuscular and subcutaneous administration. According to one embodiment, the pharmaceutical composition is for subcutaneous administration.

According to any one of the above embodiments, the pharmaceutical composition of the present invention further comprising an additional active agent. According to some embodiments the additional active ingredient is an anti-viral agent. According to some embodiments, the active agent is a protease inhibitor. According to some embodiments, the protease inhibitor is selected from darunavir, lopinavir, ritonavir and a combination thereof according to another embodiment, the active agent is selected from atazanavir (ATV), amprenavir, fosamprenavir (APV), tipranavir (TPV), indinavir, saquinavir, lopinavir/ritonavir, prezista, nelfinavir (NFV) and azidothymidine (AZT).

According to yet other embodiments, the additional active ingredient is an anti-cancer agent.

According to some embodiments, the pharmaceutical composition of the present invention further comprises lentivirus particles.

The term “active agent” refers to an agent that has biological activity, pharmacologic effects and/or therapeutic utility.

According to any one of the above embodiments, the pharmaceutical composition of the present invention is for use in treating a disease treatable with the peptide consisting of amino acid sequence WTAVQMAVFIHNFKRK (SEQ ID NO: 1) or a salt thereof. According to some embodiments, the disease treatable with a peptide of SEQ ID NO: 1 is selected from cancer and viral infection.

According to one embodiment, the disease is cancer. According to another embodiment, the disease is acquired immune deficiency syndrome (AIDS). According to one embodiment, the AIDS is caused by human immunodeficiency virus 1 (HIV-1) infection. According to one embodiment, the disease is a viral disease caused by lentivirus.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or medulloblastoma, Hodgkin's disease, Non-Hodgkin's lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes tumors, lobular carcinoma, ductal carcinoma, cancer of the pancreatic stellate cells, cancer of the hepatic stellate cells, or prostate cancer. According to some embodiments, the cancer is a solid cancer. According to one embodiment, the cancer is a colon cancer. According to another embodiment, the cancer is a pancreatic cancer.

The term “treating” a condition or patient refers to taking steps to obtain beneficial or desired results, including clinical results. Beneficial or desired clinical results include, but are not limited to, ameliorating abrogating, substantially inhibiting, slowing or reversing the progression of a disease, condition or disorder, substantially ameliorating or alleviating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and/or (e) limiting recurrence of symptoms in patients that were previously asymptomatic for the disorder(s).

According to some embodiments, treating AIDS comprises reducing a viral load. According to one embodiment, treating of AIDS comprises reducing the HIV-1 RNA count to below 1000 copies/ml, below 800 copies/ml, below 600 copies/ml, below 300 copies/ml or below 100 copies/ml.

According to some embodiments, treating AIDS comprises reducing a viral load by at least 10%, at least 20%, at least 30%, at least 40%, at least 50%, at least 60%, at least 70%, at least 80%, at least 85%, at least 90%, at least 95% or at least 97% in comparison to the baseline.

According to other embodiments, treating AIDS comprises increasing the count of CD4+ T-cells by at least 20% in comparison to the base line. According to another embodiment, treating AIDS comprises increasing the count of CD4+ T-cells by at least 30%, at least 40%, at least 50%, at least 60%, at least 70%, at least 80%, at least 90%, or at least 95% in comparison to the base line. According to a further embodiment, treating AIDS comprises increasing the count of CD4+ T-cells by at least 97% or at least 98% in comparison to the base line. According to another embodiment, treating AIDS comprises increasing the count of CD4+ T-cells 2, 2.5, 3, 4, 5 or 10 times in comparison to the base line.

The pharmaceutical composition of the present invention may be administered by any know method. The term “administering” or “administration of” a substance, a compound, an agent or a composition comprising any of them, to a subject can be carried out using one of a variety of methods known to those skilled in the art. For example, a compound or a compositing comprising said compound, can be administered, intravenously, arterially, intradermally, intramuscularly, intraperitonealy, intravenously, subcutaneously, ocularly, sublingually, orally (by ingestion), intranasally (by inhalation), intraspinally, intracerebrally, and transdermally (by absorption, e.g., through a skin duct). A compound or a composition can also appropriately be introduced by rechargeable or biodegradable polymeric devices or other devices, e.g., patches and pumps, or formulations, which provide for the extended, slow or controlled release of the compound or agent. According to one embodiment, the composition is administered intravenously. According to another embodiment, the composition is administered intramuscularly. According to a further embodiment, the composition is administered subcutaneously.

The precise dose to be employed also depends on the route of administration, and the progression of the disease or disorder, and should be decided according to the judgment of the practitioner and each patient's circumstances. Typical dosage of the INS peptide in a composition according to the present invention, is in the range 0.01 to 5 mg/kg/dose. According to some embodiments, the INS peptide is administered in the dosage of 0.01 to 2, 0.05 to 1.5, 0.1 to 1.2, 0.2 to 1, 0.4 to 0.8 0.3 to 0.7 or 0.4 to 0.6 mg/kg/dose. According to some embodiments, the composition comprises from 1 to 30 mg of the peptide INS HCl, from 2.5 to 20 mg, from 5 to 15 mg or 10 mg of the peptide INS HCl. According to some embodiments, the pharmaceutical composition is administered in the amount of 10 to 50 mg/dose, 15 to 40 mg/dose, 20 to 30 mg/dose. The composition may be administered as a single daily dose or in multiple doses. The administration schedule can be taken once-daily, twice-daily, thrice-daily, once-weekly, twice-weekly, thrice-weekly, once-monthly, twice-monthly, thrice-monthly, or any other administration schedule known to those of skill in the art. According to some embodiments, the pharmaceutical composition is administered twice a week. According to some embodiment, the composition is administered in a dose of 10 to 40 or 15 to 30 mg twice a week. The administration may be continuous, i.e., every day, or intermittently. The terms “intermittent” or “intermittently” as used herein means stopping and starting at either regular or irregular intervals. For example, intermittent administration can be administration in one to six days per week or it may mean administration in cycles (e.g. daily administration for two to eight consecutive weeks, then a rest period with no administration for up to one week) or it may mean administration on alternate days.

According to one embodiment, treatment of AIDS is affected as described in WO 2010/041241. According to some embodiment, treatment of AIDS comprises administering the composition of the present invention in combination with at least one additional active agent, for example an anti-HIV agent. According to some embodiments, the anti-HIV agent is a protease inhibitor. According to some embodiments, the protease inhibitor is selected from lopinavir, darunavir, ritonavir and a combination thereof.

According to one embodiment, treatment of AIDS comprises administering the composition of the present invention in combination with at least one protease inhibitor. According to some embodiments, the composition is administered in combination with lopinavir and ritonavir. According to some embodiments, treating comprises administering the composition of the present invention from 1 to 4 times a week in combination with 400 to 800 mg of lopinavir once a day and/or 100 to 300 mg of ritonavir once a day, wherein the dose of the INS peptide is from 0.1 to 1 mg/kg/dose. According to one embodiment, treatment of AIDS comprises administering to the subject in need of such treatment, 2 times a week, a composition comprising 0.2 to 0.4 mg/kg of INS peptide in combination with daily administration of 800 mg lopinavir and 200 mg ritonavir. According to any one of the above embodiments, the composition of the present invention is administered subcutaneously.

According to one embodiment, treating of AIDS results in reducing the HIV-1 RNA count to below 600 copies/ml. According to another embodiment, treating AIDS results in increasing the count of CD4+ T-cells by at least 80%, at least 85, at least 90%, at least 95%, or at least 98% in comparison to the base line.

According to one embodiment, co-administration of the composition of the present invention and optionally of an at least one additional active agents, is performed in a regimen selected from a single combined composition, separate individual compositions administered substantially at the same time, and separate individual compositions administered under separate schedules and include treatment regimens in which the agents are not necessarily administered by the same route of administration or at the same time. The composition of the present invention and of an additional active agent are administered in a sequential manner in either order. According to other embodiments, the composition of the present invention and of an additional active agent are administered in a substantially simultaneous manner, i.e. administered with only a short time interval between them. In some embodiments, the time interval is in the range of from 0.5 to 60 minutes. In other embodiment the co-administration is a simultaneous administration.

According to another embodiment, treatment of cancer is performed as described in WO2018215999. According to some embodiment, treatment comprises administering the composition of the present invention in combination with any other known treatment.

According to one embodiment, the composition of the present invention is administered in combination with:

(i) a linear molecule of double-stranded DNA (dsDNA) comprising long term repeat (LTR) sequences recognized by the integrating enzyme of (ii), and an integrating enzyme, capable of entering the nuclei of cells after binding to the LTR sequences of the dsDNA molecule of (i) and creating multiple double strand breaks (DSBs) in the chromosomal DNA of the cells.

According to some embodiment, the composition of the present invention is administered in combination with (i) a lentivirus particle comprising a linear molecule of double-stranded DNA (dsDNA) comprising long term repeat (LTR) sequences recognized by the integrating enzyme of (ii), (ii) an integrase, capable of entering the nuclei of cells after binding to the LTR sequences of the dsDNA molecule of (i) and creating multiple double strand breaks (DSBs) in the chromosomal DNA of the cells, and (iii) an antibody capable of binding CD24 or CD20 presented by the cancer cells.

According to some embodiments, the pharmaceutical composition is co-administered with viral particles. According to one embodiment, the viral particles are lentiviral particles. According to some embodiments, the viral particles such as lentiviral particles comprise an antibody capable of binding CD24 or CD20. According to one embodiment, the viral particles such as lentiviral particles comprise a single chain antibody directed against CD24 or CD20.

The term “integrating enzyme” as used herein refers to any enzyme capable of creating double stranded breaks (DSBs) in the chromosomal DNA of a human cell. The “integrating enzyme” may optionally be further capable of incorporating a double stranded DNA molecule into the gap formed by the DSBs. Non-limiting examples of integrating enzymes are the integrase enzymes of retroviruses, such as the integrase enzyme of HIV-1.

In certain embodiments, the term “HIV-1 integrase” as used herein relates e.g. to the protein having the UniProtKB Entry Q76353 (Q76353_9HIV1). In certain embodiments, the term “HIV-2 integrase” as used herein relates e.g. to the protein having the UniProtKB Entry D5LQ24 (D5LQ24_9HIV2). Due to the high sequence diversity in HIV genes, many other variants of HIV-1 integrase and HIV-2 integrase are further considered as “integrating enzymes” according to the present invention.

Retroviral integrase is an enzyme produced by a retrovirus (such as HIV) that enables its genetic material to be integrated into the DNA of the infected cell. Since retroviruses are rapidly and constantly changing, the exact sequence of retroviral integrase is practically impossible to follow. In certain embodiments, the integrating enzyme is an integrase enzyme selected from the group consisting of HIV-1 integrase, HIV-2 integrase, active fragments thereof, and active analogs thereof. Each possibility represents a separate embodiment of the invention.

According to some embodiments, the pharmaceutical composition is administered twice a week to provide from 10 to 40 mg of INS HCl peptide salt per dose in combination with lentiviral particles comprising a single chain antibody directed against CD24, e.g. 10⁸ to 10¹⁰ IU of the particles.

One particular such a method is described in WO2018215999.

According to some embodiments, the pharmaceutical composition according to any one of the above embodiments is a lyophilized composition.

According to another aspect, the present invention provides a solid pharmaceutical composition comprising a salt of a peptide comprising amino acid sequence TAVQMAVFIHNFKRK and a poloxamer having the structure of Formula 0

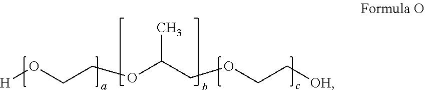

wherein a and c are each independently an integer from 50 to 120, and b is an integer from 15 to 40, and the peptide consists of 15 to 40 amino acids. According to one embodiment, the weight ratio between said salt and said poloxamer is from about 10:1 to about 1:1500.

According to one embodiment, the peptide comprises the amino acid sequence WTAVQMAVFIHNFKRK (SEQ ID NO: 1). According to one embodiment, the peptide consists of amino acid sequence WTAVQMAVFIHNFKRK (SEQ ID NO: 1, INS). According to another embodiment, the peptide consists of amino acid sequence TAVQMAVFIHNFKRK (SEQ ID NO: 2).

According to one embodiment, a and c are each independently an integer from 55 to 115, from 60 to 110, from 65 to 105, from 70 to 100 or from 75 to 90. According to another embodiment, b is an integer from 15 to 35, or from 20 to 30. According to some embodiment, a=b. According to such embodiment, the poloxamer has the structure of Formula I:

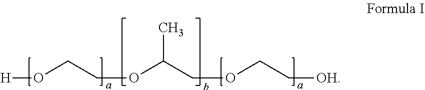

According to one embodiment, the salt is hydroxyl halide salt. According to one embodiment, the hydroxyl halide salt is selected from HCl, HBr and HI salt. According to one embodiment, the composition comprises the peptide salt INS HCl.

According to one embodiment, the present invention provides a solid pharmaceutical composition comprising a salt of peptide consisting of amino acid sequence SEQ ID NO: 1 and a poloxamer having a structure of Formula I,

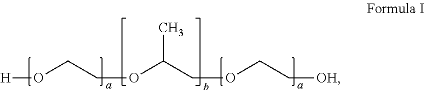

wherein a is an integer from 50 to 120, and b is an integer from 15 to 40. According to one embodiment, the weight ratio between said INS salt and said poloxamer is from about 10:1 to about 1:1500. According to another embodiment, the weight ratio between said salt and said poloxamer is from 9:1 to 1:1250, from 8:1 to 1:1100, from 7:1 to 1:800, from 6:1 to 1:600, from 5:1 to 1:400, from 4:1 to 1:200, from 3:1 to 1:100 from 2:1 to 1:50 or from 1:1 to 1:25. According to another embodiment, the weight ratio between said salt and said poloxamer is from 5:1 to 1:50, from 4:1 to 1:40, from 3:1 to 1:30, from 2:1 to 1:20 or from 1:1 to 1:10.

According to one embodiment, the poloxamer is as defined hereinabove. According to one embodiment, the poloxamer is poloxamer 188.

According to one embodiment, the peptide salt is HCl salt of INS. According to one embodiment, the present invention provides a solid pharmaceutical composition comprising an HCl salt of INS and a poloxamer 188, wherein the weight ratio between said salt and said poloxamer is from about 10:1 to about 1:1000. According to another embodiment, the weight ratio between INS HCl and poloxamer 188 is from 5:1 to 1:50, from 4:1 to 1:40, from 3:1 to 1:30, from 2:1 to 1:20 or from 1:1 to 1:10. According to one embodiment, the weight ratio between the INS HCl and the poloxamer 188 is from about 2:1 to about 1:20.

According to any one of the above embodiments, the solid pharmaceutical composition of the present invention further comprises a saccharide selected from a disaccharide and a polysaccharide. According to one embodiment, the saccharide is as defined hereinabove. According to one embodiment, the saccharide is a disaccharide. According to one embodiment, the disaccharide is lactose.

According to one embodiment, the weight ratio between the INS HCl and the lactose is from about 1:2000 to about 5:1.

According to one embodiment, the weight ratio between the INS HCl and the lactose is from about 1:1000 to about 4:1, from 1:500 to 3:1, from 1:300 to 2:1, from 1:100 to 1:1, or from 1:50 to 1:10. According to another embodiment, the weight ratio between the INS

HCl and the lactose is from 5:1 to 1:30, from 4:1 to 1:24, from 3:1 to 1:20, from 2:1 to 1:15 or from 1:1 to 1:10.

According to one embodiment, the present invention provides a solid pharmaceutical composition comprising the peptide salt INS HCl, poloxamer 188 and lactose, wherein the weight ratio between INS HCl and poloxamer 188 is between about 3:1 to 1:20 and the weight ratio between INS HCl and lactose is between about 4:1 to about 1:24.

According to one embodiment, the present invention provides a solid pharmaceutical composition comprising from 0.9 wt % to about 70 wt % of an INS salt of and a poloxamer having a structure of Formula I,

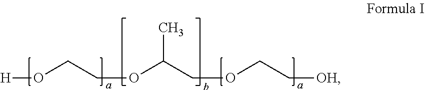

wherein a is an integer from 50 to 120, and b is an integer from 15 to 40. According to some embodiments, the weight ratio between said INS peptide salt and said poloxamer is from about 10:1 to about 1:1500. According to another embodiment, the weight ratio between said peptide salt and said poloxamer is from 9:1 to 1:1250, from 8:1 to 1:1100, from 7:1 to 1:800, from 6:1 to 1:600, from 5:1 to 1:400, from 4:1 to 1:200, from 3:1 to 1:100 from 2:1 to 1:50 or from 1:1 to 1:25. According to another embodiment, the weight ratio between said peptide salt and said poloxamer is from 5:1 to 1:50, from 4:1 to 1:40, from 3:1 to 1:30, from 2:1 to 1:20 or from 1:1 to 1:10.

According to one embodiment, the poloxamer is as defined hereinabove. According to one embodiment, the poloxamer is poloxamer 188. According to one embodiment, the salt is HCl salt of INS. According to one embodiment, the present invention provides a solid pharmaceutical composition comprising from about 4.5 wt % to about 60 wt % of the peptide salt INS HCl and poloxamer 188. According to another embodiment, the composition comprises from about 1.5 wt % to about 50 wt % of the peptide salt INS HCl. According to any one of the above embodiments, the solid pharmaceutical composition of the present invention further comprises a saccharide selected from a disaccharide and polysaccharide. According to one embodiment, the saccharide is as defined hereinabove. According to one embodiment, the saccharide is a disaccharide. According to one embodiment, the disaccharide is lactose.

According to one embodiment, the weight ratio between the peptide INS HCl and lactose is from about 1:2000 to about 5:1.

According to one embodiment, the present invention provides a solid pharmaceutical composition comprising from about 2 wt % to about 22 wt % INS HCl peptide, from about 3.5 wt % to about 63 wt % poloxamer 188 and from about 30 wt % to about 92 wt % of lactose. According to about embodiment, the solid pharmaceutical composition comprising from about 1.75 wt % to about 18.5 wt % INS HCl peptide, from about 5 wt % to about 19.5 wt % poloxamer 188 and from about 95 wt % to about 89 wt % of lactose. According to yet another embodiment, the solid pharmaceutical composition comprising from about 2 wt % to about 20 wt % INS HCl peptide, from about 8 wt % to about 9.5 wt % poloxamer 188 and from about 75 wt % to about 88 wt % of lactose.

According to one embodiment, the solid pharmaceutical composition is a lyophilized composition.

According to any one of the above embodiments, the solid pharmaceutical composition of the present invention upon a reconstitution forms a liquid pharmaceutical composition of the present invention, e.g. a parenteral pharmaceutical composition.

According to any one of the above embodiments, the solid pharmaceutical composition of the present invention upon a reconstitution comprises from about 0.1 to about 30 mg/ml of a salt of a peptide consisting of amino acid sequence WTAVQMAVFIHNFKRK (SEQ ID NO: 1), and from about 0.3 wt % to about 15 wt % of a poloxamer, wherein the pH of the composition is between 4 to about 7.5 and the poloxamer has a structure of Formula I,

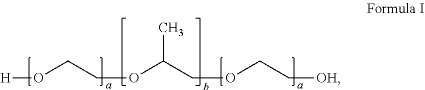

wherein a is an integer from 50 to 120, and b is an integer from 15 to 40.

According to another embodiment, the present invention provides a reconstituted solid pharmaceutical composition of the present invention. According to some embodiments, the reconstituted pharmaceutical composition is for use in treating a disease selected from cancer and AIDS.

According to another aspect, the present invention provides a method of treating a disease, the method comprises parenterally administering to a subject in need thereof an effective amount of the pharmaceutical composition comprising a peptide consisting of amino acid sequence WTAVQMAVFIHNFKRK (SEQ ID NO: 1), of the present invention. According to some embodiments, the present invention provides a method of treating a disease in a subject in need thereof, the method comprises parenterally administering a therapeutically effective amount of a reconstituted solid pharmaceutical composition of the present invention.

According to some embodiments, the disease is selected from a viral infection and cancer. According to some embodiments, the viral infection is of HIV. According to one embodiment, administration is selected from the group consisting of intravenous, intramuscular and subcutaneous administration. According to another embodiment, the disease is AIDS caused by HIV-1. According to one embodiment, the method of treating is as described in WO 2010/041241. According to one embodiment, the disease is cancer. According to another embodiment, treatment of cancer is affected as described in WO2018215999. According to some embodiments, the composition of the present invention is administered in combination with at least one additional therapeutically active agent. According to some embodiments, the active agent is a protease inhibitor. According to some embodiments, the protease inhibitor is selected from lopinavir, darunavir, ritonavir and a combination thereof.

According to some embodiments, the composition of the present invention is administered in combination with lentivirus particles, as described hereinabove or in WO 2018/215999. According to some embodiment, the lentivirus particles comprise a moiety that specifically bind a tumor antigen. According to some embodiments, the lentivirus particles comprises single chain antibody directed against CD24 or CD20.

According to some embodiment, the INS HCl peptide is administered once a week, twice a week or 3 times a week. According to some embodiment, the INS HCl peptide is administered in an amount of 0.01 to 2, 0.05 to 1.5, 0.1 to 1.2, 0.2 to 1, 0.4 to 0.8 0.3 to 0.7 or 0.4 to 0.6 mg/kg/dose. According to other embodiments, the method comprises administering from 10 to 50 mg/dose, 15 to 40 mg/dose, 20 to 30 mg/dose of INS HCl, e.g. twice a week. According to one embodiment, the INS HCl peptide is co-administered with lentivirus particles comprising single chain antibody directed against CD24.

According to another aspect, the present invention provides a pharmaceutical composition comprising from about 5 wt % to about 15 wt % of Cremophor RH40, from about 1 to about 20 mg/ml of an INS salt, and a pharmaceutically acceptable carrier. According to one embodiment, the salt is as defined herein above. According to one embodiment, the peptide salt is INS HCl. According to one embodiment, the composition comprises from 5.5 wt % to 14.5 wt %, from 6 wt % to 14 wt %, from 7 wt % to 13 wt % or from 8 wt % to 12 wt % of Cremophor RH40. According to one embodiment, the composition comprises 10 wt % of Cremophor RH40. According to another embodiment, the composition comprises from about 2 mg/ml to about 18 mg/ml, from about 4 mg/ml to about 16 mg/ml, from about 6 mg/ml to about 14 mg/ml or from about 8 mg/ml to about 12 mg/ml of the INS HCl peptide. According to one embodiment, the pH of the composition is from 4.5 to about 5.5

According to one embodiment, the present invention provides a pharmaceutical composition comprising from about 8 wt % to about 12% of Cremophor RH40, from about 5 to about 10 mg/ml of the peptide INS HCl, and a pharmaceutically acceptable carrier, wherein the pH of the composition is from about 4.5 to about 5.5.

The term “Cremophor RH 40” refers to PEG-40 castor oil or Polyoxyl 40 hydrogenated castor oil.

According to another aspect, the present invention provides a pharmaceutical composition comprising from about 10 wt % to about 30 wt % propylene glycol (PG), from about 0.3 to about 1.5 wt % Tween 80, from about 1 mg/ml to about 8 mg/ml of the peptide salt INS HCl, and a pharmaceutically acceptable carrier. According to one embodiment, the pH of the composition is from about 4.5 to about 6. According to one embodiment, the composition comprises from 10 wt % to 30 wt %, from about 12 wt % to 28 wt %, from 14 wt % to 26 wt %, from 16 wt % to 24 wt % from 18 wt % to 22 wt % of (PG). According to one embodiment, the composition comprises from 0.4 wt % to 1.4 wt %, from 0.6 to 1.2 wt % of Tween 80. According to one embodiment, the present invention provides a pharmaceutical composition comprising from 15 to 25 wt % of PG, from 0.6 to 1 wt % of Tween 80 and from 4 to 8 mg/ml of the peptide salt INS HCl.

According to another aspect, the present invention provides a hydrogen halide salt of a peptide comprising amino acid sequence selected from WTAVQMAVFIHNFKRK (SEQ ID NO: 1) and TAVQMAVFIHNFKRK (SEQ ID NO: 2), wherein said peptide consists of from 15 to 30 amino acids.

According to one embodiment, the peptide consists of the amino acid sequence WTAVQMAVFIHNFKRK (SEQ ID NO: 1, INS). According to another embodiment, the peptide consists of amino acid sequence TAVQMAVFIHNFKRK (SEQ ID NO: 2). According to one embodiment, the hydrogen halide is selected from HCl, HBr, and HI. According to one embodiment, the present invention provides an hydrochloride salt of the peptide INS. According to another embodiment, the present invention provides an HBr salt of the peptide INS. According to yet another embodiment, the present invention provides an HCl or HBr salt of a peptide consisting of the amino acid sequence

(SEQ ID NO: 2)
TAVQMAVFIHNFKRK.

According to another embodiment, the present invention provides a pharmaceutical composition comprising a peptide salt according to any one of the above embodiments.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term “about”, when referring to a measurable value such as an amount, a temporal duration, and the like, is meant to encompass variations of +/−10%, or +/−5%, +/−1%, or even +/−0.1% from the specified value.

Having now generally described the invention, the same will be more readily understood through reference to the following examples, which are provided by way of illustration and are not intended to be limiting of the present invention.

### EXAMPLES

The present invention provides stock formulations of INS peptide at the concentration of up to 10 mg/ml. These formulations are stable upon administration to physiological liquids, e.g. they may be diluted in PBS up to 10 times and maintain clear solution upon dilution without precipitation of the peptide.

### Materials and Methods

The peptide denoted INS, having the sequence WTAVQMAVFIHNFKRK (SEQ ID NO: 1) was prepared as described in WO 2010/041241, stored at −20° C., and used at concentration of 2-10 mg/ml.
Chemicals that were used for preparation of the formulations of the present invention are summarized in Table 1.

TABLE 1
Chemicals used in development of the formulations

| Name | Manufacture | Batch No. |
|---|---|---|
| Trifluoroacetic acid | Alfa Aesar | U10D816 |
| Acetonitrile (ACN) | Merck | I0896730 725 |
| PEG 400 | Sinopharm Chemical | 20170717 |
| | Reagent Co. Ltd | |
| Propylene glycol (PG) | SIGMA-ALDRICH | MKBZ1609V |
| Ammonium bicarbonate | N/A | T067101 |
| HP-β-CD | Kunshan RSK | 170801 |
| SBE-β-CD | ROQUETTE | N/A |
| Phosphate-buffered saline (PBS) | Thermo scientific | NZD1121 |
| Cremophor EL | BASF | 4126664760 |
| 1N NaOH | STA-DFR | N/A |
| POLOXAMER P188 (Kolliphor ® P 188) | BASF | N/A |
| Solutol HS 15 | Sigma-Aldrich | BCBN9145V |
| Cremophor RH 40 | SIGMA | BCBG5390V |
| Tween 80 | SPECTRUM CHENICAL | 3DK0061 |
| L-Arginine | N/A | 13040003 |
| 28% Ammonoium hydroxide | Alfa Aesar | 10183704 |
| D-glucose | ENOX | 20110801 |
| Anhydrous Lactose | Deceling | N/A |
| D-Mannitol | Roquette | E519K |
| D-Sorbitol | SCR | F20110509 |
| 1% Tween 80 | STA-DFR | FR00110-1- |
| | | 180327-01 |
| 1% Poloxamer P188 | STA-DFR | FR00110-1- |
| | | 180327-03 |
| 20% HP-β-CD (Hydroxy propyl beta | STA-DFR | N/A |
| Cyclodextrin) | | |
| Sodium acetate Trihydrate | Sinopharm Chemical | 20150109 |
| | Reagent Co. Ltd | |
| 9.5% Lactose + 0.8% Poloxamer P188 | STA-DFR | FR00110-5- |
| (w/v) | | 180509-01 |

### Preparation of Buffers and Solutions

0.1M citirate buffer pH 6: 11.5 ml 0.1M citric acid (0.1M) were mixed with 88.5 ml sodium citrate (0.1M).
50 mM phosphate buffer, pH 8.0: 25 mL of the 0.2M potassium phosphate solution were mixed with 24 ml of 0.2M NaOH.
50 mM borate buffer, pH 9.0: 12 ml 0.2M NaOH were added to 25 ml 0.2M boric acid and potassium chloride solution.
0.1M NH₄HCO₃: about 99.87 mg NH₄HCO₃ was weighed and dissolved by 12.6 ml purified water.
1% Tween 80 (w/w): to 200 mg of Tween 80 were added 19.80 mL of purified water and mixed well.
5% poloxamer P188 (w/w): 1000 mg of poloxamer P188 were placed in a 20 ml volumetric flask and 19.00 mL of purified water were added and mixed well.
10% HP-β-CD (w/w): 2000 mg of HP-β-CD were placed in a 20 ml volumetric flask and add 18.00 mL of purified water were added and mixed well.
9.5% Lactose+0.8% Poloxamer P188 (w/v): 2373 mg of lactose and 200 mg of poloxamer P188 were weighed in a 25 mL volumetric flask, 15 ml of purified water were added to dissolve completely; then the solution was diluted to volume (25 ml) at room temperature. The 9.5% lactose+0.8% poloxamer P188 was used for making peptide solution formulation.
The stability of the solubilized INS peptide was tested using high performance liquid chromatography (HPLC) protocols presented in Tables 2 and 3.

TABLE 2
HPLC method for detection of INS peptide (60 min)

| Column details | Germini C18 (150 * 4.6 mm, 5 μm), 110A | | |
|---|---|---|---|
| Column temperature | 40° C. | | |
| Mobile phase A | 0.1% TFA in water (v/v) | | |
| Mobile phase B | 0.075% TFA in ACN | | |
| Flow rate | 1 mL/min | | |
| | | % Mobile | % Mobile |
| Gradient profile | Time (mins) | Phase A | Phase B |
| | 0 | 95 | 5 |
| | 8 | 95 | 5 |
| | 43 | 60 | 40 |
| | 45 | 95 | 5 |
| | 60 | 95 | 5 |

| | |
|---|---|
| Detector wavelength | 200 nm |
| Injection volume | 10 μL |
| Needle wash solvent | H₂O |
| Dilution | ACN/H₂O 1:1 |

TABLE 3
Modified HPLC method for detection of INS peptide (15 min)

| Column details | Germini C18 (150 * 4.6 mm, 5 μm), 110A | | |
|---|---|---|---|
| Column temperature | 40° C. | | |
| Mobile phase A | 0.1% TFA in water (v/v) | | |
| Mobile phase B | 0.075% TFA in ACN | | |
| Flow rate | 1 mL/min | | |
| | | % Mobile | % Mobile |
| Gradient profile | Time (mins) | Phase A | Phase B |
| | 0 | 90 | 10 |
| | 10 | 60 | 40 |
| | 10.1 | 60 | 40 |
| | 15 | 90 | 10 |

| | |
|---|---|
| Detector wavelength | 200 nm |
| Injection volume | 10 μL |
| Needle wash solvent | H₂O |
| Dilution | ACN/H₂O 1:1 |

### Example 1. HPLC Characterization of the INS Peptide

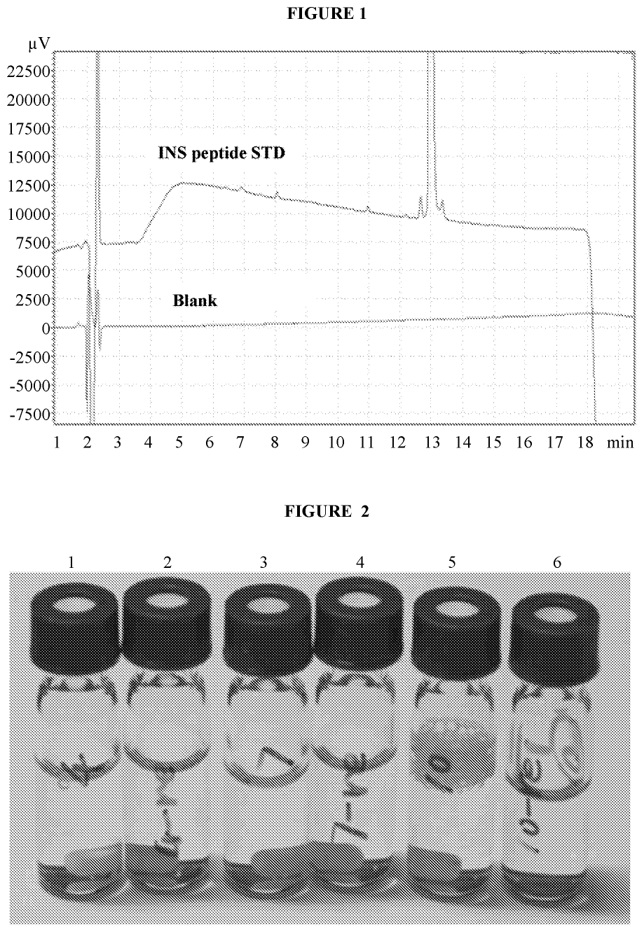

INS peptide was dissolved in pure water to form a solution of about 0.5 mg/ml and analyzed by HPLC (modified method—Table 3). The purity results, indicating a purity of about 98.71% are presented in Table 4 and FIG. 1.

TABLE 4
HPLC elution of INS peptide

| Retention Time | Area % | S/N |
|---|---|---|
| 12.156 | 0.062 | 0.27 |
| 12.648 | 0.405 | 2.03 |
| 12.958 | 98.708 | 460.41 |
| 13.333 | 0.825 | 2.48 |

### Example 2. Preliminary Solubility in Different pH Values

Buffers pH solubility and stability plays an important role in drug discovery and development. Knowledge of buffer stability profiles helps in modifying pre-formulation and formulation as well as the molecular structure to improve physicochemical properties and in selecting more suitable molecules for subsequent development.

### Material and Methods

Analytical HPLC-MS was performed using an Agilent 1260 series Liquid Chromatograph/Mass Selective Detector (MSD, Single Quadrupole) equipped with an electrospray interface and a UV diode array detector. Analyses were performed on an ACE C8, 50×3.0 mm, 3 μm column, with an isocratic 0.1% formic acid/ACN (19%) eluent, at a rate of 1 ml/min over a period of 5 minutes. UV absorption was measured at 220 nm and 215-395 nm. MS analysis was performed with ESI (electron spray ionization) with a positive ionization method.
PBS isotonic buffer was prepared from 1 tablet of Sigma Phosphate buffer saline P4417, in 200 ml of deionized water (according to product reconstitution instructions). Acetate isotonic buffers were prepared from sodium acetate/acetic acid in water in 150 mM concentration. NaOH 50% solution was used to adjust the pH.
NaOAc solution was prepared by dissolving 1.23 gr in 100 ml water. Acetic acid solution was prepared by adding 945 μl of acetic acid to 100 ml water.
INS HCl salt was used directly in the buffers at concentration of 5 mg/ml.
The samples were further diluted to 1 mg/ml before analyzing by HPLC. The samples were prepared by dilution of 80 μl of reaction solution with 320 μl of MeOH.
Each sample was filtered through PTFE 0.45 um filter vial.
The samples were run in LC-MS and data used for calculation concentration in buffer.
All injections for stability assay were performed using 20 μl due to low peak intensity with 2 μl. Final concentration was calculated accordingly.

### Results

The results are presented in Tables 5-7. It can be seen that in acetate buffers having pH 4 and pH 6 the INS peptide showed better stability at 5° C. compared to the samples at 25° C. It can be seen that the assay for both cases was already very low (about 70%) at time zero. In PBS buffer having pH 7.4 the INS peptide had a no solubility at all and a white precipitate was observed.

TABLE 5
INS stability in acetate buffer pH = 4 at 5° C. and at 25° C.

| | 5° C. | | 25° C. | |
|---|---|---|---|---|
| | Concentration | | Concentration | |
| Time | calculated from | | calculated from | |
| (hours) | LC-MS data (mg/ml) | Assay % | LC-MS data (mg/ml) | Assay % |
| 0 | 0.646 | 66.84 | 0.646 | 66.87 |
| 24 | 0.602 | 62.32 | 0.640 | 66.25 |
| 168 | 0.599 | 62.00 | 0.355 | 36.75 |

TABLE 6
INS stability in acetate buffer pH = 6 at 5° C. and at 25° C.

| | 5° C. | | 25° C. | |
|---|---|---|---|---|
| | Concentration | | Concentration | |
| Time | calculated from | | calculated from | |
| (hours) | LC-MS data (mg/ml) | Assay % | LC-MS data (mg/ml) | Assay % |
| 0 | 0.729 | 73.49 | 0.729 | 73.49 |
| 24 | 0.530 | 53.43 | 0.520 | 52.42 |
| 168 | 0.438 | 44.15 | 0.218 | 21.98 |

TABLE 7
INS stability in PBS buffer pH = 7.4 at 5° C. and at 25° C.

| | | Concentration | |
|---|---|---|---|
| Time | Area | calculated from | |
| (hours) | (mAU*S) | LC-MS data (mg/ml) | Assay % |
| 0 | 201.90 | 0.030 | 2.91 |
| 24 | Not observed | — | — |
| 168 | Not observed | — | — |

### Example 3. Preliminary Solubility Test

Solubility of different INS peptide salts was measured in several co-solvents and in water.

### INS Trifluoroacetate (TFA)

INS TFA salt was dissolved in water and DMSO up to a concentration of 10 mg/ml, however upon dilution in PBS, the INS peptide precipitated.

### INS Acetate

INS acetate salt was soluble in water and completely insoluble in DMSO and PBS. It could be dissolved in low pH, but precipitated when the pH was raised above 4.

### INS HCl

The INS peptide could be dissolved in water and formed a solution with opalescence; the pH was 2.6, which could be ascribed to the fact that the peptide is an HCl salt. Although INS TFA showed moderate solubility, it is less recommended to be used for parenteral product, thus, the HCl salt was selected for further studies. The compound showed moderate solubility in PG (a clear solution was obtained), while it shows poor solubility in PEG400 and ethanol. Table 8 shows the solubility result of the INS HCL peptide in co-solvents and water. It should be noted that the INS HCL peptide is practically insoluble in PBS namely in physiological conditions.

TABLE 8
Solubility results of the INS HCL peptide in co-solvents/water

| Co-solvents/Buffers | Approximate Solubility (mg/mL) |
|---|---|
| PBS | Not soluble |
| PEG400 | <5 |
| Ethanol | <5 |
| Propylene glycol (PG) | 5~10(clear solution in 5mg/ml) |
| DMSO | ≤10 |
| H₂O | >10 (slightly opalescence) pH 2.60 (10 mg/ml) |

### Example 4. Preliminary Screening of Formulations

The target concentration of the formulation was first set at 10 mg/mL, which may be diluted up to 10 times with PBS. Dilution in PBS predicts the solubility of the peptide in physiological conditions and in particular in blood and interstitial fluid. Table 9 lists the results obtained with initial formulations. The results indicated that clear solutions of peptide formulation could be obtained if small amounts of co-solvents or surfactants were added. However, the results of a dilution test in PBS showed that opalescence was observed in most of the formulations. Appearance of opalescence may indicate that peptide aggregates or precipitates in PBS.

TABLE 9
Content and dilution results of preliminary formulations

| | Formulation components | Appearance | final pH | Dilution in PBS |
|---|---|---|---|---|
| A1 | water | opalescence | 2.60 | opalescence |
| A2 | 10% PG, 10% Cremophor EL, and | clear | 3.23 | slight opalescence |
| | 10%, pH8 buffer¹ | | | particle of about 1 μm |
| A3 | 15% PG, 15% pH6 buffer¹ | gel state | N/A | N/A |
| A4 | 4% SB-β-CD, 10% pH8 buffer¹ | white | N/A | N/A |
| | | precipitation | | |
| A5 | 25% PG,, 10% pH8 buffer¹ | gel state | N/A | N/A |
| A6 | 20% PG, 10% pH8 buffer¹ | clear | 3.24 | slight opalescence |
| A7 | 10% Cremophor EL, 10% pH8 buffer¹ | Clear | 3.23 | slight opalescence |
| A8 | 10% PEG400, 10% Cremophor EL, | Clear | 3.65 | slight opalescence |
| | 10% pH8 buffer¹ | | | |
| A9 | 10% PEG400, 10% PG, 10% pH8 | Clear | 3.44 | slight opalescence |
| | buffer¹ | | | |
| A10 | 10% PEG 400, 10% Cremophor EL, | Clear | 3.64 | slight opalescence |
| | 10% pH9 buffer¹ | | | |
| A11 | 10% PEG 400, 10% Cremophor EL, | Clear | 4.90 | slight opalescence |
| | 10% 0.1M NH₄HCO₃ | | | |
| A12 | 80% water, 20% 0.1M NH₄HCO₃ | Clear | 6.25 | slight opalescence |

¹pH6 buffer- 0.1M citirate buffer,
pH8 buffer- 50 mM phosphate buffer,
pH9 buffer- 50 mM borate buffer

The results with 10 mg/mL concentration were not satisfactory. Opalescence was observed in redispersibility test and the pH was very low because of the existence of HCl.

### Example 5. Formulation Screening

Based on the results obtained in Example 4, the concentration of the INS HCl peptide was reduced to 5 mg/ml or 2.5 mg/ml in order to prepare a clear formulation, which would maintain clear when diluted in PBS. The pH of all formulations was adjusted to be between 5 to 6 with about 4 μl 1N NaOH to meet the parenteral formulation requirement. Unless stated explicitly, the carrier in all formulation was water. The content of the tested formulations and results of their dilution in PBS are summarized in Table 10.

TABLE 10
Content and dilution results for the tested formulations

| | | Conc. | | | Redispersion in |
|---|---|---|---|---|---|
| | Formulation Components | (mg/ml) | Appearance | Final pH | PBS |
| F1 | 1% Tween 80 | 5 | clear | 5.80 | slight opalescence |
| F2 | 20% PG | 5 | clear | 6.13 | slight opalescence |
| F3 | 10% SBE-β-CD | 5 | white | N/A | N/A |
| | | | precipitation | | |
| F4 | 5% Poloxamer P188 | 5 | clear | 5.61 | clear |
| F5 | 10% HP-β-CD | 5 | clear | 6.11 | slight opalescence |
| F6 | 10% Cremophor EL | 5 | clear | 5.49 | slight opalescence |
| F7 | 10% Cremophor RH40 | 5 | clear | 5.48 | clear |
| F8 | 10% Solutol HS15, 4.5% | 5 | clear | 5.58 | slight opalescence |
| | Poloxamer P188 | | | | |
| F9 | 10% Cremophor EL, 4.5% | 5 | clear | 5.82 | slight opalescence |
| | Poloxamer P188 | | | | |
| F10 | 20% PG, 0.8% Tween 80 | 5 | clear | 5.59 | clear |
| F11 | 0.5% Poloxamer P188 | 5 | clear | 7.63 | slight opalescence |
| F12 | 0.5% Poloxamer P188 | 8 | slight | 7.05 | slight opalescence |
| | | | opalescence | | |
| F13 | 0.5% Poloxamer P188 | 10 | slight | 6.99 | slight opalescence |
| | | | opalescence | | |
| F14 | 0.5% Poloxamer P188 | 2.5 | clear | 7.41 | clear |
| F15 | 5% Poloxamer P188 | 10 | clear | 5.90 | clear |
| F16 | 5% Poloxamer P188 | 8 | clear | 6.38 | clear |
| F17 | 5% Poloxamer P188 | 6 | clear | 6.99 | clear |
| F18 | 10% Cremophor RH40 | 10 | clear | 5.57 | slight opalescence |
| F19 | 10% Cremophor RH40 | 8 | clear | 5.80 | slight opalescence |
| F20 | 10% Cremophor RH40 | 6 | clear | 5.52 | slight opalescence |
| F21 | 10% Cremophor EL | 2.5 | clear | N/A | slight opalescence |
| F22 | 1% poloxamer | 5 | clear | 6.08 | slight opalescence |
| | | | | | (0.33)* |
| F23 | 1% poloxamer P188, 0.1% | 5 | clear | 5.8 | slight opalescence |
| | Tween 80 | | | | (0.02)* |
| F24 | 10% PG, 0.9% poloxamer | 5 | clear | 5.72 | slight opalescence |
| | P188, 0.09% Tween 80 | | | | (0.16)* |
| F25 | 10% Glycerol, 0.9% | 5 | clear | 5.64 | slight opalescence |
| | poloxamer P188 | | | | (0.09)* |
| F26 | 20% PG, 10% cremophor | 10 | clear | 5.66 | opalescence |
| | EL | | | | |
| F27 | 20% PG, 10% cremophor | 5 | clear | 5.88 | slight opalescence |
| | EL | | | | |
| F28 | 20% PG, 10% solutol HS15 | 10 | clear | 5.50 | opalescence |
| F29 | 20% PG, 10% solutol HS15 | 5 | clear | 5.62 | slight opalescence |
| F30 | 20% PG, 0.8% Tween 80 | 10 | clear | 6.02 | opalescence |
| F31 | 20% PG, 4% poloxamer | 5 | clear | 5.95 | slight opalescence |
| | P188 | | | | |
| F32 | 20% PG, 10% HP-β-CD | 5 | clear | 5.98 | slight opalescence |

*value in (mg/ml) means the concentration of INS HCl peptide after filtration of the diluted solution through a 0.45 μm filter; the concentration was measured by HPLC; the estimated concentration should be equal to 0.5 mg/ml.

As can be seen in Table 10 and FIG. 2, formulation F4, F7, F10 and F14-17 showed the best performance. These formulations were clear and remained clear after dilution with PBS. Formulations F4, F7 and F10 were further tested for stability using HPLC.

### Example 6. Stability of INS HCl Peptide in Formulation A6

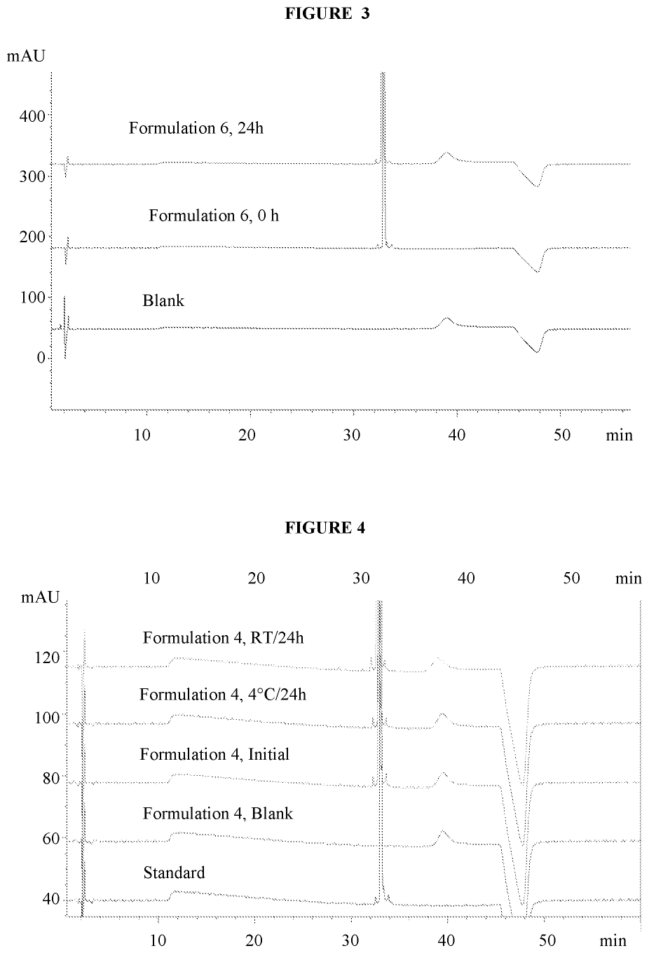

Stability of INS in Formulation A6 at concentration of 10 mg/ml for 1 day was investigated. Formulation A6 was prepared according to Table 9. The samples were diluted with water prior to HPLC analysis. The recovery of INS from formulation 6 is almost 100% after 24 hours. No impurities were detected. The purity results are listed in Table 11 and FIG. 3.

TABLE 11
Peaks of INS in formulation F6 as determined by HPLC

| | Area % | | | | | |
|---|---|---|---|---|---|---|
| Retention time (min) | 32.32 | 32.40 | 32.89 | 33.22 | 33.51 | 33.68 |
| Initial | 0.387 | 0 | 98.125 | 0.779 | 0.177 | 0.532 |
| 24 h | 0.437 | 0.141 | 97.937 | 0.77 | 0.237 | 0.478 |

### Example 7. Stability of INS HCl Peptide in Formulations F4, F7 and F10

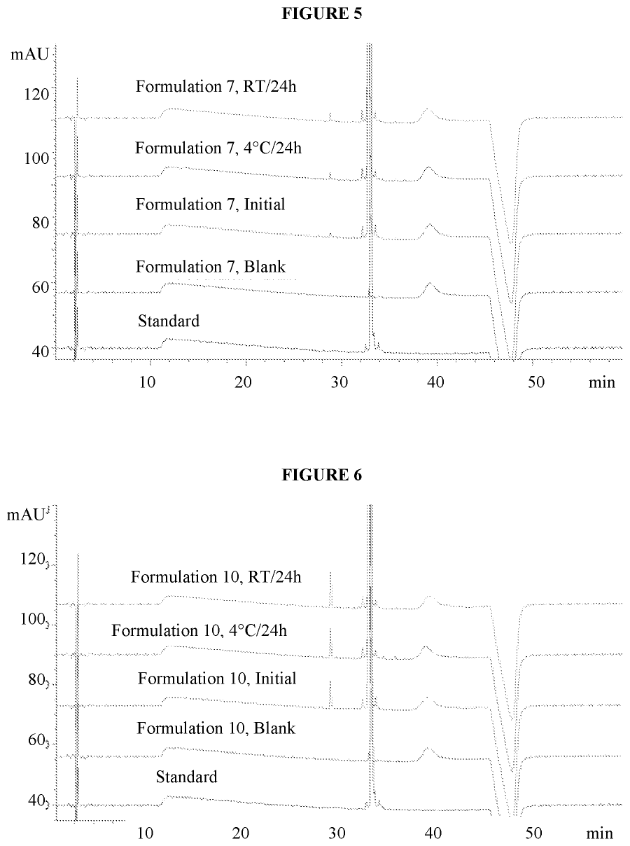

To investigate the stability of formulation F4, F7 and F10 they were stored at ambient temperature and at 4° C. Samples were taken out at time zero (initial) and after 24 hours and diluted 10 times with water prior to HPLC analysis. The results were compared to the standard peptide 0.5 mg/ml in water (STD). The results are presented in Tables 12-14 and FIGS. 4-6. Based on these results, a new impurity (RRT=0.88) was observed. INS HCL demonstrated high stability in all formulations, while it seems that formulation F4 provides a little bit higher stability than the other two.

TABLE 12
Stability results (%) of Formulation F4

| | Relative Retention Time (RRT) | | | | | | |
|---|---|---|---|---|---|---|---|
| # | 0.88 | 0.95 | 0.98 | 1.00 | 1.01 | 1.02 | 1.03 |
| STD | ND | ND | 0.32 | 98.79 | 0.64 | ND | 0.25 |
| Initial | 0.08 | ND | 0.36 | 98.38 | 0.64 | 0.07 | 0.47 |
| 4° C./24 h | 0.07 | ND | 0.42 | 98.52 | 0.69 | ND | 0.30 |
| RT/24 h | 0.16 | 0.06 | 0.52 | 97.51 | 1.01 | 0.22 | 0.53 |

TABLE 13
Stability results (%) of Formulation F7

| | RRT | | | | | | | |
|---|---|---|---|---|---|---|---|---|
| # | 0.88 | 0.98 | 0.99 | 1.00 | 1.01 | 1.02 | 1.03 | 1.09 |
| STD | ND | 0.32 | ND | 98.79 | 0.64 | ND | 0.25 | ND |
| Initial | 0.20 | 0.38 | 0.05 | 98.19 | 0.65 | 0.06 | 0.47 | ND |
| 4° C./24 h | 0.25 | 0.40 | 0.06 | 98.24 | 0.67 | ND | 0.26 | 0.12 |
| RT/24 h | 0.30 | 0.43 | ND | 98.34 | 0.67 | ND | 0.27 | ND |

TABLE 14
Stability results (%) of Formulation F10

| | RRT | | | | | | | | | |
|---|---|---|---|---|---|---|---|---|---|---|
| # | 0.88 | 0.97 | 0.98 | 0.99 | 1.00 | 1.01 | 1.02 | 1.03 | 1.05 | 1.09 |
| STD | ND | ND | 0.32 | ND | 98.79 | 0.64 | ND | 0.25 | ND | ND |
| Initial | 1.18 | ND | 0.37 | ND | 97.29 | 0.64 | 0.08 | 0.44 | ND | ND |
| 4° C./24 h | 1.26 | ND | 0.45 | 0.05 | 97.02 | 0.63 | 0.05 | 0.27 | 0.10 | 0.17 |
| RT/24 h | 1.52 | 0.07 | 0.51 | ND | 96.94 | 0.69 | ND | 0.27 | ND | ND |

ND—Not Detected

### Example 8. Optimization of Formulations

Based on previous solubility and stability results, it seems that the opalescence in PBS may indicate aggregation of the peptide, which may be related with several factors such as pH, ionic strength, oxidation of the peptide, etc. To expand the repertoire of the formulations tested, additional excipients were used. The target concentration of the INS HCl peptide was set at 5 mg/mL. The additional excipients were added in stepwise manner, and then the mixtures were stirred at 25° C. for 5 mins to facilitate dissolution. The formulations were then diluted 10 folds in PBS and tested by HPLC. Unless stated explicitly, the carrier in all formulation was water. Table 15 summarized the content of formulations and their dilution stability.

TABLE 15
Content and dilution results for the tested formulations

| | Formulation | Conc. | | | Redispersion |
|---|---|---|---|---|---|
| | Components | (mg/ml) | Appearance | Final pH | in PBS |
| F33 | 1% poloxamer P188, | 5 | clear | 5.66 | slight |
| | 5% Lactose | | | Add 2 ul of 6% | opalescence |
| | | | | NH₄OH | |
| F34 | 1% poloxamer P188, | 5 | clear | 5.67 | Clear (0.46)* |
| | 10% Lactose | | | Add 2 ul of 6% | |
| | | | | NH₄OH | |
| F35 | 20% PG, 0.8% | 5 | clear | 6.08 | slight |
| | poloxamer P188, 0.08% | | | Add 1.0 mg of | opalescence |
| | L-Arginine | | | L-Arginine | |
| F36 | 1% poloxamer P188, | 5 | clear | 6.32 | slight |
| | 0.1% L-Arginine | | | Add 1.0 mg of | opalescence |
| | | | | L-Arginine | |
| F37 | 0.1% L-Arginine | 5 | clear | 6.24 | opalescence |
| | | | | Add 1.0 mg of | |
| | | | | L-Arginine | |
| F38 | 20% HP-β-CD | 5 | clear | 6.68 | slight |
| | | | | Add 1.0 mg of | opalescence |
| | | | | L-Arginine | |
| F39 | 20% PG, 0.8 1% Tween | 5 | clear | 6.83 | slight |
| | 80 | | | Add 1.0 mg of | opalescence |
| | | | | L-Arginine | |
| F40 | 1% poloxamer P188, | 5 | clear | 5.80 | slight |
| | 5% Glucose | | | Add 2 ul of 6% | opalescence |
| | | | | NH₄OH | |
| F41 | 1% poloxamer P188, | 5 | clear | 5.78 | slight |
| | 5% Mannitol | | | Add 2 ul of 6% | opalescence |
| | | | | NH₄OH | |
| F42 | 1% poloxamer P188, | 5 | clear | 5.75 | slight |
| | 5% Sorbitol | | | Add 2 ul of 6% | opalescence |
| | | | | NH₄OH | |

*value in (mg/ml) means the concentration of INS after filtration of the diluted solution through a 0.45 μm filter; the concentration was measured by HPLC; the calculated concentration equals to 0.5 mg/ml.

It was found that formulation F34 showed the best performance. The formulation remained clear after dilution in PBS with no particles detected.

### Example 9. Scale Up

Formulation F34 was scaled up; two additional formulations, having a pH of 5.5 and 4.0 were prepared as described in Table 16 below.

TABLE 16
Scale-up of Formulation 34.

| | Weigh of INS | Drug-loading | | Acetate | Poloxamer | Lactose | Volume |
|---|---|---|---|---|---|---|---|
| Formulation | peptide (mg) | (mg/mL) | pH | (μM) | P188 (%) | (mM/%) | (mL) |
| F43 | 50 | 5 | 5.5 | 100 | 1 | 2.8/9.5 | 10 |
| F44 | 50 | 5 | 4.0 | 100 | 1 | 2.8/9.5 | 10 |

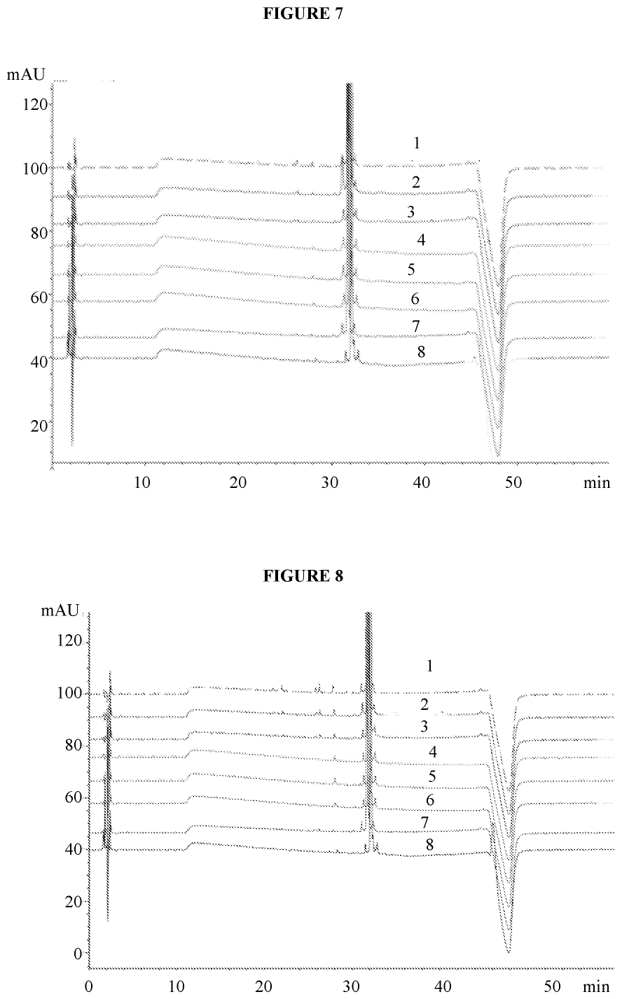

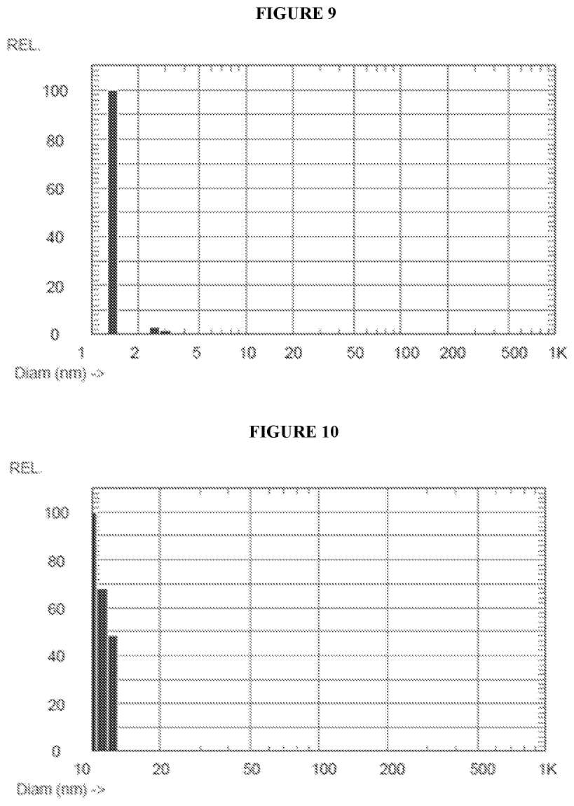

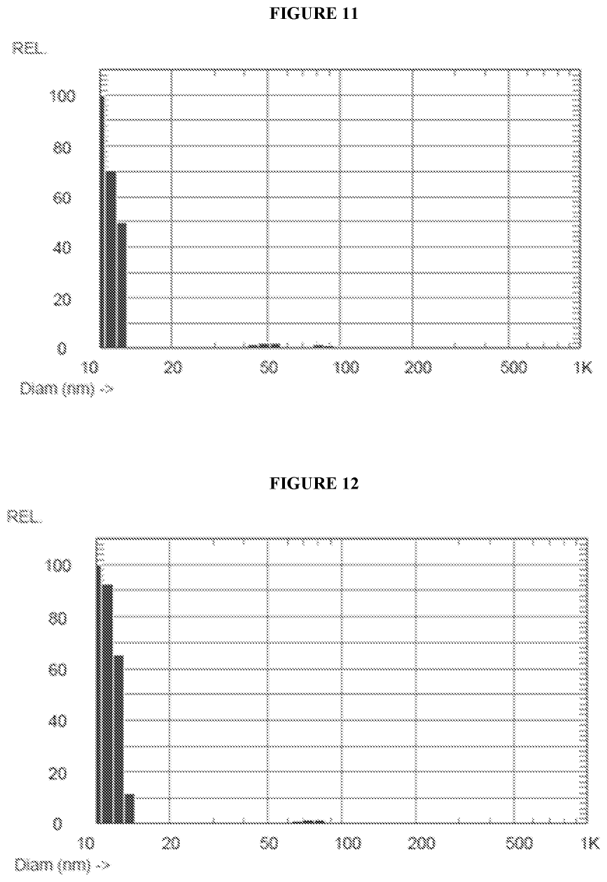

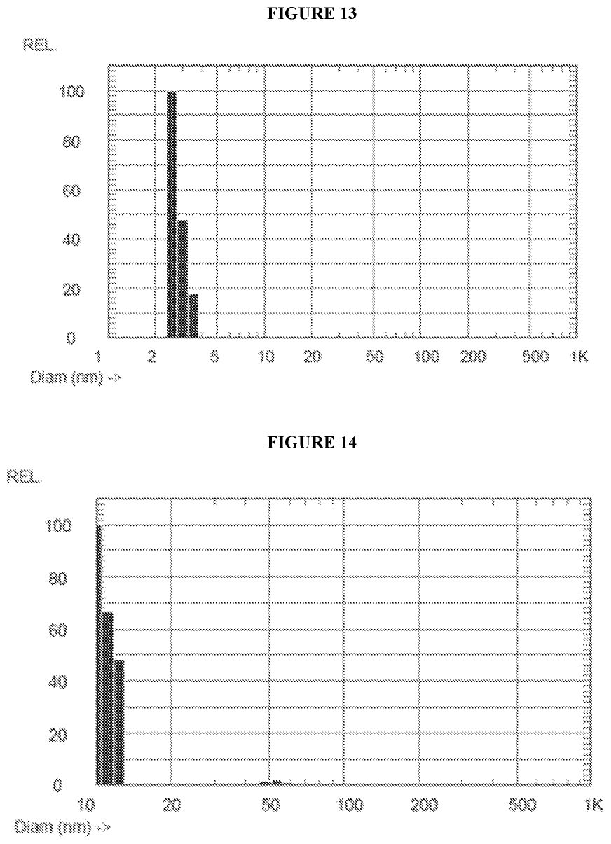

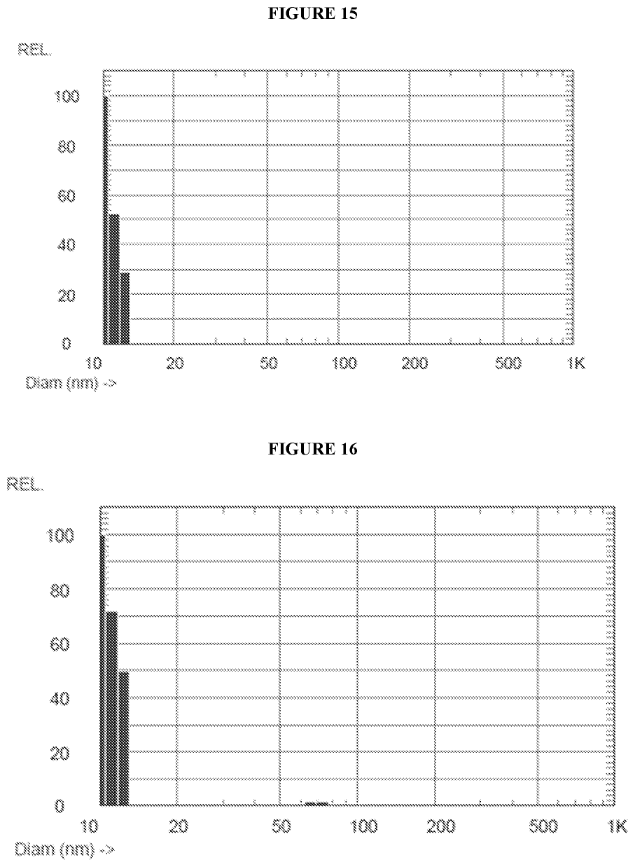

Formulation F43 and F44 were prepared and tested for peptide stability after storage at 25° C. and 5° C. Stability in freeze-thaw conditions were also assessed placing samples in −20° C., thawing for about 1-2 h in room temperature and then returning to −20° C. (this process was counted as one time/cycle). Appearance, pH, and osmolality were recorded and the concentration was analyzed by HPLC to evaluate possible degradation. The samples were taken at time points: 0 h, 24 h and 1 week. The formulations were filtered through 0.45 μm filters and the % recovery after filtration were measured at 1 week. At last, the aggregation and purity of the INS HCl peptide were examined by dynamic light scattering (DLS). The results are presented in Tables 17-19 and FIGS. 7-16.

TABLE 17
physical properties of formulations F43 and F44.

| | Formulation | | | | Purity | Recovery |
|---|---|---|---|---|---|---|
| | condition | Appearance | pH | Osmolality | % | % |
| F43 | initial | Clear solution | 5.58 | 372 | 98.23 | 95.15 |
| | 5° C.-1 d | Clear solution | 5.66 | 378 | 97.91 | N/A |
| | 25° C.-1 d | Clear solution | 5.65 | 377 | 97.89 | N/A |
| | −20° C.-1 d, freeze and | Clear solution | 5.65 | 371 | 97.78 | N/A |
| | thaw 3 times | | | | | |
| | 5° C.-1 w | Clear solution | 5.72 | 363 | 98.07 | 95.93 |
| | 25° C.-1 w | Clear solution | 5.76 | 363 | 95.85 | 96.70 |
| | −20° C.-1 w, freeze and | Clear solution | 5.71 | 366 | 97.81 | 95.29 |
| | thaw 3 times | | | | | |
| F44 | initial | Clear solution | 3.95 | 367 | 98.17 | 97.90 |
| | 5° C.-1 d | Clear solution | 3.97 | 362 | 98.22 | N/A |
| | 25° C.-1 d | Clear solution | 3.95 | 371 | 98.18 | N/A |
| | −20° C.-1 d, freeze and | Clear solution | 3.98 | 366 | 98.26 | N/A |
| | thaw 3 times | | | | | |
| | 5° C.-1 w | Clear solution | 4.05 | 355 | 97.76 | 97.68 |
| | 25° C.-1 w | Clear solution | 4.05 | 358 | 97.01 | 97.24 |
| | −20° C.-1 w, freeze and | Clear solution | 4.10 | 361 | 97.36 | 97.81 |
| | thaw 3 times | | | | | |

TABLE 18
HPLC analysis of formulation F43

| | RRT | | | | | | |
|---|---|---|---|---|---|---|---|
| Formulation 43 | 0.82 | 0.88 | 0.98 | 0.99 | 1.00* | 1.01 | 1.02 |
| initial | ND | ND | 0.42 | ND | 98.23 | 0.76 | 0.60 |
| 5° C./24 h | ND | 0.13 | 0.70 | ND | 97.91 | 0.76 | 0.33 |
| 25° C./24 h | ND | 0.16 | 0.89 | ND | 97.87 | 0.85 | 0.42 |
| −20° C./Lyopholized/24 h | ND | 0.12 | 0.79 | ND | 97.78 | 0.88 | 0.44 |
| 5° C./1 W | 0.11 | ND | 0.53 | ND | 98.07 | 0.83 | 0.46 |
| 25° C./1 W | 0.13 | ND | 2.34 | 0.13 | 95.85 | 1.18 | 0.37 |
| −20° C./Lyopholized /1 W | 0.22 | 0.20 | 0.50 | ND | 97.81 | 0.82 | 0.46 |

*INS peptide
ND—Not Detected

TABLE 19
HPLC analysis of formulation F44

| | RRT | | | | | | | |
|---|---|---|---|---|---|---|---|---|
| Formulation 44 | 0.69 | 0.81 | 0.82 | 0.88 | 0.98 | 1.00* | 1.01 | 1.02 |
| initial | ND | ND | 0.11 | ND | 0.36 | 98.17 | 0.78 | 0.59 |
| 5° C./24 h | ND | ND | ND | ND | 0.40 | 98.22 | 0.68 | 0.46 |
| 25° C./24 h | ND | ND | ND | ND | 0.41 | 98.15 | 0.66 | 0.47 |
| −20° C./Lyopholized/24 h | ND | ND | ND | ND | 0.35 | 98.26 | 0.68 | 0.47 |
| 5° C./1 W | ND | ND | 0.19 | 0.23 | 0.39 | 97.76 | 0.94 | 0.50 |
| 25° C./1 W | 0.13 | 0.12 | 0.24 | 0.31 | 0.63 | 97.01 | 1.07 | 0.48 |
| −20° C./Lyopholized/1 W | 0.21 | 0.21 | 0.40 | 0.34 | 0.36 | 97.36 | 0.63 | 0.49 |

*INS peptide
ND—Not Detected

It can be clearly seen from these results that no change in the appearance, pH, osmolality was observed for formulations F43 and F44 after 1 week.
In the DLS experiments, several populations of particle size were detected. Particles with the size of around 10 nm may indicate micelle formulation of the poloxamer 188 and some observed particles with the size of >40 nm may be correlative to aggregation of peptide molecules. Nevertheless, no more than 2% of such aggregation particles (>20 nm) were observed. The % recovery of the INS HCL peptide as measured by HPLC, was above 95% after filtration through 0.45 μm filters. The peptide aggregation or absorption to the filters may explain the slightly lower recovery value in comparison to the theoretical one. It was shown that INS HCL is relatively stable at 5° C. in F43 formulation, but showed about 3-3.5% reduction when maintained at 25° C. condition for 1 week. Similar trend was observed for F44; some degradation (about 1-2%) was observed at 25° C. and −20° C. freeze-thaw condition for 1 week.

### Example 10. Optimization and Preparation of a Lyophilized Formulation

To obtain an isotonic solution having desired osmolality of about 300-330 mOsm/kg, the poloxamer percentage was reduced to 0.8% and the lactose percentage was reduced to 9.5%. The solution formulation F45 was prepared in a 40 ml clear glass vial according to Table 20. The pH was adjusted by NH₄OH. After preparation, the solution was filtered through a 0.22 μm filter (PTFE) and pre-freeze at −50° C. dry ice bath for 2 hours, and then the samples were transferred into the freeze-dryer.

TABLE 20
Preparation of isotonic formulation.

| | INS | Drug-loading free | | | | |
|---|---|---|---|---|---|---|
| | peptide | INS peptide | | Poloxamer | Lactose | Total Volume |
| | (mg)¹ | (mg/mL) | pH | P188 (%) | (%) | (mL)² |
| F45 | 57.06 | 5 | 5.05 | .08 | 9.5 | 10 |

¹The correction factor for calculating the INS peptide percentage was 1.14 (1/88.11%)
²The INS HCl peptide was weighed and then a 10 ml solution containing 9.5% Lactose and 0.8% Poloxamer P188 was added, then about 20 μl NH₄OH solution was added to adjust pH.

The lyophilization parameters of final product batch no. FR00110-6-180514-01 are listed in Table 21. After preparation, the lyophilized powder was stored and sealed in 40 mL clear glass vial with plastic stopper and stored at −20° C. refrigerator. Yield was calculated to be about 90.62% (solid mass value was used to roughly calculate the yield).

TABLE 21
The parameters of processing lyophilized formulation

| | Temperature | Vacuum | | Weight of final | |
|---|---|---|---|---|---|
| Step | (° C.) | Degree (pa) | Time (h) | product (mg) | Yield % |
| 1 | −50 | 1 | 5 | 985.11 | 90.62 |
| 2 | −30 | 10 | 3 | | |
| 3 | −20 | 20 | 2 | | |
| 4 | −10 | 20 | 2 | | |
| 5 | −5 | 10 | 2 | | |
| 6 | 0 | 10 | 2 | | |
| 7 | 5 | 15 | 2 | | |
| 8 | 10 | 15 | 2 | | |
| 9 | 20 | 15 | 2 | | |
| 10 | 30 | 10 | 1 | | |

### Example 11. Stability Study of Lyophilized Formulation

The physical and chemical stability of lyophilized powder obtained in Example 10 was investigated. The lyophilized powder was stored at −20° C. At each time point, about 218 mg of the lyophilized material was accurately weighed into a 4 mL glass vial and reconstituted completely with 2 mL of water after 2 mins at approximate target concentration of 5 mg/mL (INS peptide free base) at 0 h, 1 d and 7 ds. Formulations before filtration were used as a control (initial state) at each time point. After 2 minutes, the reconstituted formulations were filtered through a 0.22 μm filter (PTFE). At the time point: 0 h, 24 hours and 7 days appearance, pH and osmolarity were tested and the solutions were analyzed by HPLC for the presence of degradation. The recovery percent after filtration was also calculated.
To investigate short-term in-use stability, additional 218 mg of lyophilized material was reconstituted, and the solution was stored at room temperature for 4 hours. The samples were then analyzed by HPLC to examine the purity and the recovery after filtration through a 0.22 μm filter (PTFE).

### Results

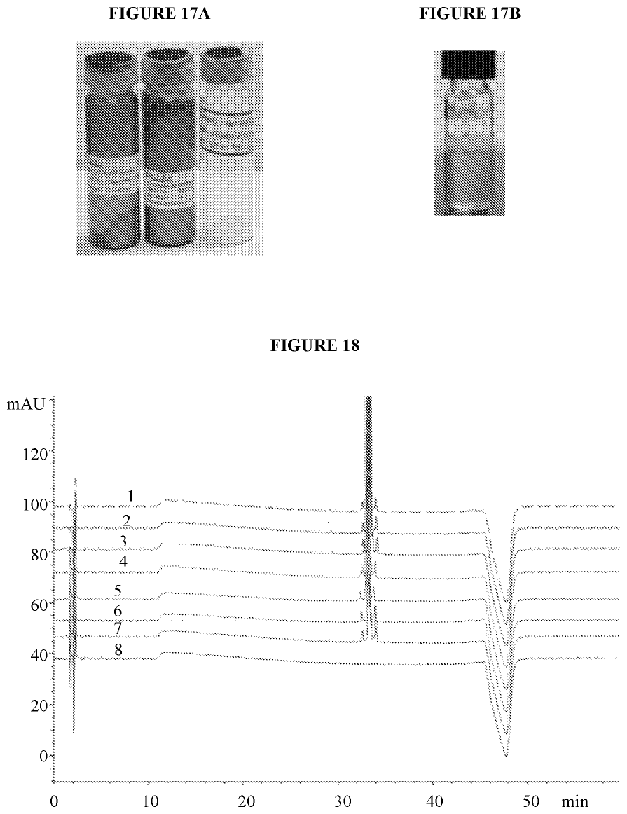

The results summarized in Tables 22-24 and FIGS. 17-18 indicate that the lyophilized formulation showed good physical and chemical stability; no obvious changes in appearance, osmolality, or purity were observed for the reconstituted lyophilized powder. The pH was slightly lower than in the initial control solution, i.e. before lyophilization. The recovery of the INS HCl peptide from the reconstituted lyophilized formulation was more than 95% after filtrating through 0.22 μm filters (PTFE). The reconstituted INS HCl peptide was stable at room temperature for at least 4 hours showing no significant difference in comparison to control. PTFE filters were used in further experiments as it was found out that about 30% of the peptide was adsorbed to PES filters.

TABLE 22
Stability of lyophilized formulation

| | | | | | | Recovery % |
|---|---|---|---|---|---|---|
| | | | | | | (peak after |
| | | | | Concentration of | | filtration/peak |
| | | | | INS peptide free | Purity | before |
| Condition | Appearance | pH | Osmolality | base (mg/mL)¹ | % | filtration) |
| Control² | Clear solution | 5.05 | 325 | 5.00 | 96.98 | 100.00 |
| Control | Clear solution | | | 4.81 | 97.06 | 96.36 |
| filtered | | | | | | |
| 0.22 μm | | | | | | |
| Reconstituted | Clear solution | 4.56 | 311 | 4.83 | 97.66 | 100.00 |
| initial | | | | | | |
| Reconstituted | Clear solution | | | 4.57 | 97.76 | 94.55 |
| initial | | | | | | |
| filtered | | | | | | |
| 0.22 μm | | | | | | |
| Reconstituted | Clear solution | 4.59 | 314 | 4.79 | 98.06 | 100.00 |
| 1 day | | | | | | |
| Reconstituted | Clear solution | | | 4.79 | 97.98 | 99.97 |
| 1 day filtered | | | | | | |
| −0.22 μm | | | | | | |
| Reconstituted | Clear solution | 4.60 | 313 | 4.88 | 98.08 | 100.00 |
| 7 days | | | | | | |
| Reconstituted | Clear solution | | | 4.62 | 97.99 | 94.59 |
| 7 days filtered | | | | | | |
| −0.22 μm | | | | | | |

¹The correction factor for calculating the INS percentage was 1.14 (1/88.11%), for 1 ml reconstituted solution, it contains about 5.7 mg INS peptide, 8 mg poloxamer 188, and 95 mg lactose.
²The peptide solution before lyophilization (pH adjusted to 5.0).

TABLE 23
HPLC analysis of lyophilized-reconstituted formulation

RRT

| Condition | 0.88 | 0.98 | 1.00* | 1.01 | 1.02 |
|---|---|---|---|---|---|
| Reconstituted initial | ND | 0.54 | 97.66 | 0.95 | 0.85 |
| Reconstituted initial | ND | 0.51 | 97.76 | 0.98 | 0.75 |
| filtered 0.22 μm | | | | | |
| Reconstituted 1 day | ND | 0.52 | 98.06 | 0.77 | 0.65 |
| Reconstituted 1 day | ND | 0.49 | 97.98 | 0.88 | 0.65 |
| filtered −0.22 μm | | | | | |
| Reconstituted 7 days | 0.11 | 0.44 | 98.08 | 0.73 | 0.64 |
| Reconstituted 7 days | 0.12 | 0.48 | 97.99 | 0.73 | 0.71 |
| filtered −0.22 μm | | | | | |

*INS peptide
ND—Not Detected

TABLE 24
The stability results of reconstituted solution at room temperature for 4 h

| | | | | | | Recovery % |
|---|---|---|---|---|---|---|
| | | | | | | (peak after |
| | | | | Conc. of INS | | filtration/peak |
| | | | | peptide free | Purity | before |
| Condition | Appearance | pH | Osmolality | base (mg/mL)* | % | filtration) |
| Reconstituted | Clear solution | 4.55 | 312 | 4.73 | 97.97 | 100.00 |
| solution 4 h at | | | | | | |
| room Temp | | | | | | |
| Reconstituted | Clear solution | | | 4.56 | 97.91 | 96.36 |
| Solution, 4 h at | | | | | | |
| room Temp, | | | | | | |
| 0.22 μm | | | | | | |
| filtered | | | | | | |

*The correction factor for calculating the INS percentage was 1.14 (1/88.11%), for 1 ml reconstituted solution, it contains about 5.7 mg INS HCl peptide, 8 mg poloxamer 188, and 95 mg lactose.

### Example 12. In Vivo Efficacy Study

### Aim

Examination of cancer treatment (together with lentiviral particles targeting to tumor antigens) in tumor mice model.

### Study Variables and End Points

1. Mortality—every day
2. Tumor measurements—twice a week
3. Tumor weight post mortem
4. Tissue preservation—kept in formalin or were snap freezed and kept at −80° C.

### Materials and Methods

### Test Items:

### I. INS

INS HCl peptide having the amino acid sequence WTAVQMAVFIHNFKRK
Physical Description: White to off-white powder

### Assay (NPC): 88.11%

### Correction Factor: 1.135 (1/88.11%)

### Storage Conditions: −10° C. to −30° C.

### Supplier: WuXi AppTec (Wuhan) Co., Ltd.

On “Day 1” stocks of 10 mg/ml (11 mg/ml—with correction factor of 1.1) INS in formulation or in PBS was prepared by dissolving 110 mg of INS in 10 ml of a vehicle solution (1% poloxamer 9% lactose), adjusting pH to about 5 with NaOH and completing the volume with the formulation up to 10 ml. The solution was vortexed and filtered using Nylon 0.22 μm filter. The content of the stock formulation is as described in Table 25.

TABLE 25
content of stock formulation.

| Raw Material | Mg/10 ml | Function | Lot No. | Manufacturer |
|---|---|---|---|---|
| INS peptide | 110* | Active ingredient | 31-18-0034 | Polypeptide |
| Poloxamer 188 | 100 | Solubilizing and | K49589112 | Merck |
| | | stabilizing agent | | |
| Lactose anhydrous | 900 | Diluent | 101FX50 | DFE pharma |
| Hydrochloric acid | QS | pH adjuster | NA | NA |
| (HCl) | | | | |
| Sodium hydroxide | QS | pH adjuster | BCBQ3682V | Fluka |
| (NaOH) | | | | analytical |
| Water for injection | 10 ml | Solvent | 16K14BA1A | Baxter |

*calculated per correction factor of 1.1 (Assay 88.11%)

Serial dilutions were made to obtain formulation comprising 5 mg/ml, 2.5 mg/ml, 0.97 mg/ml and 0.49 mg/ml by diluting the stock formulation with formulation comprising 1% poloxamer and 9% lactose.
As a control, INS HCL dissolved in PBS (at pH of about 5) was used.
The concentration of INS HCl in the stock solution and in the diluted solutions were tested by Nanodrop. The stability of the stock formulation was assessed as well. The results are provided in Table 26.

TABLE 26
Concentration of formulations

| | Prepared/diluted | A280 | Achieved |
|---|---|---|---|
| ID # | concentrations | (Abs) | Concentration |
| 1 | 5 mg/ml | 13.062 | 4.7 mg/ml |
| | t = 0 | 13.095 | 4.7 mg/ml |
| | 5 mg/ml | 12.883 | 4.6 mg/ml |
| | 2 weeks stability | 13.122 | 4.7 mg/ml |
| 2 | 2.5 mg/ml | 6.645 | 2.4 mg/ml |
| | | 6.647 | 2.4 mg/ml |
| 3 | 1 mg/ml | 2.443 | 0.9 mg/ml |
| | | 2.478 | 0.9 mg/ml |
| 4 | 0.5 mg/ml | 1.33 | 0.5 mg/ml |
| | | 1.338 | 0.5 mg/ml |
| 5* | 0.5 mg/ml (sample #1 | 1.301 | 0.5 mg/ml |
| | diluted 1:10 in PBS) | 1.342 | 0.5 mg/ml |

*The final formulation after dilution is 0.5 mg/ml INS peptide in 0.1% Poloxamer and 0.9% Lactose.

### II. Lentiviral Particles

The Lentiviral particles were manufactured at Sirion Biotech
GFP expressing lentiviral particles with packaging containing a single chain antibody directed against CD24 as N-terminal fusion to VSVG
Lot #KA17134_L4634-LT24_V_2 (empty)—5×10¹⁰ IU/ml
Lentiviral particles were diluted to 1*10⁸/200 μl in PBS.

### Tumor Mice Model:

Two models of cancer were generated: lung cancer and pancreatic cancer.
About 5×10⁶ of 1975 human lung cancer cells per 0.1 ml PBS per mouse were injected to male 7 weeks old athymic nude mice (n=30). The cells were injected, subcutaneously at one site on the back of the mice.
About 5×10⁶ PANC-1 human pancreatic cancer cells per 0.1 ml PBS per mouse were injected to male 7 weeks old athymic nude mice (n=22). The cells were injected, subcutaneously at one site on the back of the mice.
When tumors were palpable (˜0.3-0.5 cm³), the mice were randomly divided into three groups (Table 27) and the viruses and/or peptides were injected, twice a week for two weeks. The dose injected to different groups are presented in Table 28.

TABLE 27
Group allocation

| | |
|---|---|
| Group | # of mice |

Lung cancer

| | | |
|---|---|---|
| 1 | PBS + Formulation | 7 |
| 2 | LV 10⁸ + INS (1.25 mg) | 7 |
| 3 | LV 10⁸ + INS (2.5 mg) | 7 |

Pancreatic cancer

| | | |
|---|---|---|
| 4 | PBS + Formulation | 7 |
| 5 | LV 10⁸ + INS (1.25 mg) | 7 |
| 6 | LV 10⁸ + INS (2.5 mg) | 7 |

TABLE 28
Dose calculation to different test groups.

| | | | | | Dose | | | |
|---|---|---|---|---|---|---|---|---|
| | | | | | solution | | | |
| | | | Dose | Dose | (based | | | |
| | | | volume | solution | on | | | |
| | | Dose | (1000/ | free | purity | | | No. |
| Test | Group | level | 35*0.1) | base | 1.1) | Per 35 g | | of |
| Item | # | mg/kg | ml/kg | mg/ml | mg/ml | ml | mg | mice |
| INS | 2 | 1.25 | 2.9 | 0.43 | 0.49 | 0.1 | 0.043 | 7 |
| | 5 | 1.25 | 2.9 | 0.43 | 0.49 | 0.1 | 0.043 | 7 |
| INS | 3 | 2.5 | 2.9 | 0.86 | 0.97 | 0.1 | 0.097 | 7 |
| | 6 | 2.5 | 2.9 | 0.86 | 0.97 | 0.1 | 0.097 | 7 |

### Experimental Procedure

The first day of the study was defined as ‘Day 1’. The animals were administrated IP with either Vehicle (PBS or Formulation), lentiviral particles and SC with test items according to Table 27 on days 1, 4, 8 and 11.
Body weight and tumor volume were checked on days 3, 7, 11, 14 and 17 (the latter was measured with a caliper). At the end of the experiment, on day 17, the mice were anesthetized and then sacrificed, and the tumors were removed, as well as lung, spleen, kidney and liver.

### Results

### Efficacy

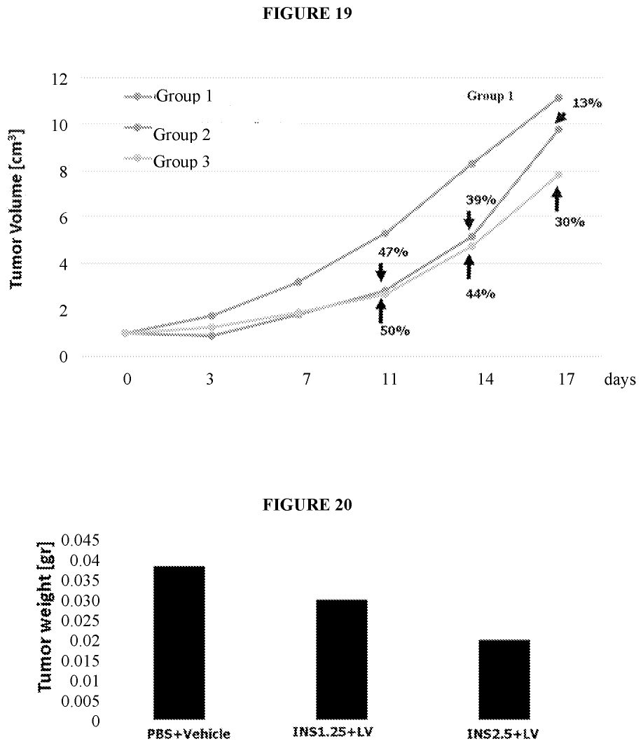

As can be seen from FIG. 19, growth of lung cancer tumors in groups administrated with full treatment, lentiviral particles and INS peptide (1.25 mg/kg or 2.5 mg/kg), was inhibited by 50% at day 11, relative to control. At the end of the experiment, on day 17, treatment with INS 1.25 mg/kg showed better results than with INS 2.5 mg/kg.
As for pancreatic cancer, weight of tumors decreases in a dose dependent manner, 25% or 50% reduction was observed in groups administered with lentiviral particles and INS (1.25 mg/kg or 2.5 mg/kg, respectively), relative to control as shown in FIG. 20.
Mortality: No animal was found dead or in morbid state during this study.
Body weight: Mice gained weight as expected, with no differences between groups.
Tumor measurements: As the tumor volume of the vehicle group increased above the ethical limits, the study was arrested.

### Conclusions:

Based on the above findings, INS HCL and lentiviral particles targeted specifically to tumors, showed a decrease of up to 50% in growth of tumors in lung cancer and pancreatic cancer models.
This study examined for the first time SC injection of the INS peptide with the new formulation. As can be seen in FIGS. 19 and 20 SC injection of INS is both safe and effective in this model.

### Example 13. Phase I/IIa Clinical Trial

### Study Type and Goal

A phase I/IIa pilot—Proof of Concept, dose escalation study to evaluate safety, dose, tolerability and preliminary efficacy of INS peptide to reduce viral load in HIV patients. This is a phase I/IIa pilot—Proof of Concept, dose escalation study.

### Primary Objective

To determine the dose of INS peptide that is most closely associated with a low rate of any grade Severity Harm suspected adverse reaction within 24 hours after the INS peptide administration.

### Secondary Objectives:

To evaluate the safety of the therapy as measured by blood tests and physical examination
To obtain a preliminary estimate of efficacy as measured by monitoring the rates of disease response at day 35 and 42 (±3 days).
To evaluate duration of response at six months and 1-year post administration.

### Multiple Ascending Dose (MAD):

The overall goal was to identify the dose of INS peptide for treating HIV patients. Treatment escalating consecutive multiple subcutaneous (SC) administration doses 0.05, 0.1, 0.2, 0.3 and 0.4 mg/kg body weight INS HCL peptide in the formulations of the present invention (2.5, 5, 10, 15, and 20 mg of INS peptide administration respectively, considering 50 kg person);

### Study Design

The study consists of three periods: screening, treatment and follow up as described below.

### Screening

Inclusion/exclusion criteria, blood chemistry, hematology, coagulation, urinalysis and vital signs are used to assure medical fitness to participate in the study.

### Inclusion Criteria:

1. Male and female 18 up to 65 years,
2. HIV patients confirmed by serology (Ab for HIV-1/HIV-2) and HIV-1/HIV-2 RNA testing.
3. Viral load <400,000 copies/mL.
4. >200 CD4 cells/mm³.
5. Willingness to undergo at least 10 visits including bi-weekly SC INS administrations; for a total duration of 4-6 weeks, as well as unscheduled visits, if required.

### Exclusion Criteria:

1. Any female patient who either is pregnant, intends to become pregnant, or is currently breastfeeding (all women of child-bearing age will be questioned and informed by the study physician regarding these criteria and requested to use adequate contraception).
2. Total neutrophil count of <1,000 u/L, Hemoglobin <9.0 gm/dl or platelet count of <75,000 u/L; serum creatinine >1.5 mgh/dl, dire ct serum bilirubin >85 μmol/l, AST or ALT >2.5 times ULN.
3. Positive result for HBV or HCV.
4. Administration of HAART—Highly Active Antiretroviral Therapy or any 12 weeks prior HIV treatment.
5. Any clinically significant Grade 3 or 4 laboratory abnormality according to the Division of AIDS (DADS) grading scale.
6. Severe morbidity (clinically, psychiatric and behaviour) such as severe Ischemic or Congestive Heart Failure, non-controlled diabetic patients, renal insufficiency, liver cirrhosis or any disease judged by the investigator to influence the study results.
7. Any significant acute illness within 1 week before the first administration of investigational medication on this study.
8. Any immunomodulating therapy (including interferon), systemic steroids, or systemic chemotherapy within 4 weeks before Day-0.
9. Patients with malignant cancer.
10. Current enrolment in an investigational drug or device study or participation in such a study within 30 days of entry into this study.
11. Any potential significant allergy or hypersensitivity to any excipient in the Gammora formulation.

### Exit Criteria:

1. HAART treatment administration during the study conduct (until termination visit)
2. Viral Load >1 log from baseline
3. Any clinically significant Grade 3 or 4 laboratory abnormality according to the Division of AIDS (DADS) grading scale.
4. Physical examinations.
5. Blood test

### Treatment

Three cohorts of 3-6 patients (per cohort) received twice a week the Multiple Ascending Doses (MAD*) for up to 8 weeks to evaluate safety, clinical dose and preliminary efficacy for a duration of up to 6 weeks.
Interim analysis was performed to decide on the safety of the low dose and continue to a higher dose SC administration.
Study Duration: 1 year including screening, treatment and post treatment follow up

### Follow-Up

2 weeks from baseline follow-up visit to evaluate safety, tolerability (drop-out rate due to side effects) and preliminary efficacy.
Additional follow-up visits will take place after 42 days (±5 days), six months (±7 days) and one year (±10 days) from baseline.

### Tested Item and Dose

INS-HCl (Gammora™) having the sequence WTAVQMAVFIHNFKRK (SEQ ID NO: 1 was manufactured by “Polypeptide” with purity of 98.3%.
The INS HCl is formulated as a sterile, non-pyrogenic isotonic aqueous solution for SC administration. The formulation comprises 1% poloxamer 188 and 9% lactose anhydrous at pH about 5.
Dose and route of administration: 0.05, 0.1, 0.2, 0.3 and 0.4 mg/kg body weight. The formulation was administered SC, twice weekly up to 8 weeks.
In cases of safety issues (excluding neutropenic fever, infections, and haemorrhages caused by HIV infection, occurred during any treatment course with INS or within 24 (±2) of INS administration) the INS dose is reduced by half, in the following administration.

### Dose Level Assignment

### Consecutive Cohorts of Multiple Ascending Dose (MAD):

Cohort 1: Three to six (3-6) patients, each one received twice a week the decided MAD* for up to 8 weeks (up to 16 doses per patient). Subcutaneous dose: LOW; 0.05, mg/kg, with escalation to 0.2 mg/kg body weight at week 2-4.
Cohort 2: Three to six (3-6) patients, each one received twice a week the decided MAD* for up to 8 weeks (up to 16 doses per patient). Subcutaneous dose: MEDIUM; 0.1 mg/kg, with escalation to 0.3 mg/kg body weight at week 2-4.
Cohort 3: Three to six (3-6) patients, each one received twice a week the decided MAD* for up to 8 weeks (up to 16 doses per patient). Subcutaneous dose: HIGH; 0.2 mg/kg, with escalation to 0.4 mg/kg body weight at week 2-4.
Interim analysis was performed to decide on the safety of all cohorts' doses and continue to a higher dose, up to 0.2 mg/kg for Cohort 1, up to 0.3 mg/kg for Cohort 2 and up to 0.4 mg/kg for Cohort 3, SC administration.

### Optional Part II of the Study.

According to the mechanism of action of known drugs, first an increase in viral load (VL) is expected followed by a decrease. If INS works accordingly, protease inhibitor may be combined with the peptide treatment to allow significant redaction of the viral load to level of “under-detected”.
The contemplated Protease Inhibitor is a drug combination Darunavir+Ritonavir (DRV/r) in dosage of 800/100 mg once daily or lopinavir 800 mg and ritonavir 200 mg once a day.

### Monitoring Adverse Events

Patients are monitored for adverse events (AE) of the INS peptide such as rash, acute allergic reaction, bronchospasm, respiratory distress, and acute vascular leak syndrome. If a severe administration related reaction occurs, the drug administration is stopped.
Blood chemistry, hematology, coagulation, and urinalysis are repeated on Days 1, 4, 8, 11, 15, 18, 22, 25 at the clinic visits. Follow-up visits are performed at day 35, 42 ((±3 days) six (6) months (±1 week) and one (1) year (±1 week).
Any suspected adverse event after the 1^{st} INS peptide administration within 24 hours. Grade Severity Harm within 42 days after the 1^{st} INS peptide administration.

### Visits Schedule

Visit 1: (up to 3 days before Baseline) Screening and Enrolment. (Medical history, Clinical examination, lab, serology and RNA tests).
Patients are prospectively enrolled to the study during a routine clinic visit, after meeting inclusion and exclusion criteria.
Visit 2 (day 1): Once all inclusions/exclusions criteria are verified, physical examination and blood tests re performed to obtain baseline values. Beginning of INS peptide administration, then within 2 hours from drug administration all safety tests to be repeated.
Visit 3-16: INS peptide administration, physical examination and blood tests are performed
Visit 17-20: Post INS peptide administration safety follow-up: can be done by telephone contact.
Visit 21: Post INS peptide administration safety follow-up. Blood chemistry, hematology, coagulation, and urinalysis will be repeated on.

### Endpoints

### Safety Endpoint

The primary safety endpoint is the frequency, severity, and duration of side effects. Number of participants with treatment-related adverse events as assessed by CTCAE v4.03, are evaluated though study completion.
Main adverse events evaluation is based on DADS Toxicity criteria (Division of AIDS DADS Table for Grading the Severity of Adult and Paediatric Adverse Events, Corrected Version 2.1):
Viral load
Lymphopenia/Neutropenia
Immuno-reaction over reaction/cytokine storm
Infection surrounding the administration site
Liver function (lab tests)
Kidney function (lab tests)

### Tolerability Endpoint

Incidence of withdrawal (drop-out rate) due to side effects is evaluated throughout the study.

### Exploratory Endpoint (Preliminary Efficacy)

Effectiveness of Gammora assessed by reduced Viral load (by 0.5 LOG; Viral Suppression).
1. Mean Change in CD4+ Cell Count as a Measure of Efficacy and Safety.
2. Reduced viral protein p24 antigen.
3. Functional Assessment of HIV Infection (FAHI) questionnaire.

### Overall Study Duration

Total study duration is approximately 370 days from signing of informed consent to last on day 365 post-INS peptide administration. Approximately 3 weeks to recruit and complete 9-18 per-protocol—patients. 10 weeks to complete last enrolled patient last drug administration, 1 year for last patient last visit (LPLV) and additional 2 weeks for study closure.

### Results

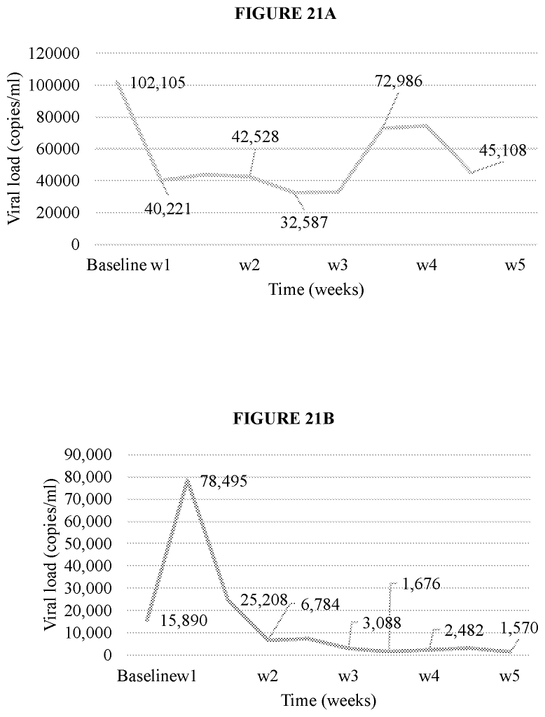

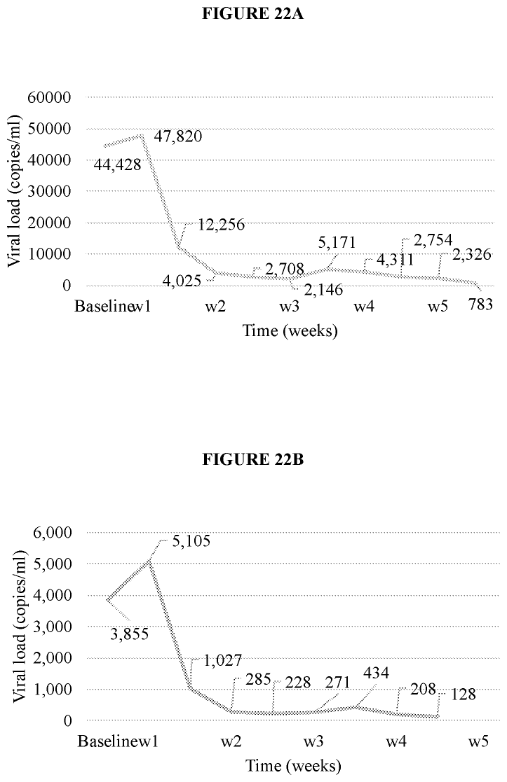

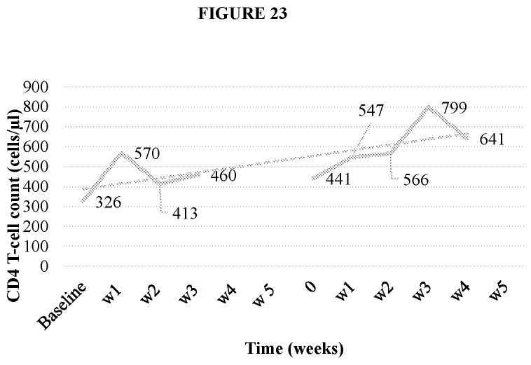

Preliminary results showed that the peptide INS HCL in a formulation according to some embodiments of the present invention, is safe and well tolerable. Patients demonstrated through 5 weeks of the experiment that the treatment with the subcutaneous administration of INS using the composition of the present invention is safe, non-toxic and well tolerable, exhibit no side effects and no harm to immune system (monitored by CD4 cells) meeting its primary endpoint. In addition, the preliminary results indicated treatment efficacy as measured by decrease in viral load in HIV patients. Most patients achieved significant reduction in viral load for up to 90% from baseline during the first 5 weeks of SC administration, meeting its exploratory endpoint. Representative examples of viral load in two different patients are presented in FIGS. 21A and 21B.
An additional short-term part II study was conducted in which a combination of INS with protease inhibitor was administered. In this study, patients were treated for 5 weeks with a combined treatment of the subcutaneous composition as previous (0.2-0.4 mg/kg twice a week) and commercially available lopinavir 800 mg plus ritonavir 200 mg daily. The control group received only lopinavir 800 mg plus ritonavir 200 mg for additional 5 weeks. According to the results, combined-treated patients demonstrated a significant viral suppression and reached reduction in HIV-1 RNA count to below 300 copies/mL, namely up to 99% reduction in viral load from baseline as shown in FIGS. 22A and 22B representing two different patients. In addition, patients treated with INS presented a significantly increase of CD4 T-cell count, up to 97% relative to baseline and improved immune recovery in comparison to the control group that showed no change as shown in FIG. 23.

### Example 14. Clinical Trial in Human Subjects to Test the Safety and Efficacy of INS HCL Peptide in Cancer Treatment

### Treatment Objectives:

To evaluate the safety, tolerability and preliminary anti-tumor activity of INS peptide (SEQ ID NO: 1), in combination with humanized anti-CD24-lentiviral (LV) particles administered to a cancer patient diagnosed with soft tissue sarcoma, after failing first and second-line therapy. Cancer cells from a biopsy taken from the cancer patient prior to the treatment were positive for CD24 (by staining) therefore, the patient is eligible for treatment targeted toward CD24.

### Treatment Endpoints:

### Primary Endpoints:

Number and frequency of treatment-related Adverse Events (SE) and Serious Adverse Events (SAE) and laboratory data

### Secondary Endpoints:

Objective tumor response
A reduction in primary tumor size to evaluate the therapeutic effectiveness of a specific short-term treatment using Computed Tomography (CT) or Magnetic Resonance Imaging (MRI). Size of the primary tumor is compared to the size of the tumor after last treatment dose.

### Additional Data to be Evaluated:

Progression free survival: The length of time during and after the treatment, that the patient lives with the disease but it does not get worse.

### Treatment Design:

Patient with advanced or metastatic cancer positive to membranal CD24, after failing first and second-line therapy are selected by the physician to receive the compassionate treatment. The patient receives a full explanation concerning the treatment and provide a signed informed consent form. The physician thoroughly assesses the patient's medical history and eligibility for receiving the treatment. An eligible patient receives a 4-week treatment of anti-CD24-LV together with INS peptide in combination with second-line gemcitabine chemotherapy plus NAB paclitaxel and other standard of care therapies as per hospitals standards.
Anti-CD24-LV treatment (I.M, 2×10⁹ IU/1 ml, twice or three times per week) is administered along with INS peptide (S.C, 20 mg per administration) during a treatment period of 6 weeks. After last treatment dose (6 weeks), the patient followed; if improvement is observed the treatment is continued, otherwise, the treatment is stopped.
Safety related parameters and preliminary efficacy parameters are evaluated throughout the treatment and follow-up period.

### Composition of the Product

1. INS peptide 10 mg/ml (20 mg per administration) for nonconsecutive days SC administration. INS peptide is produced by Polypeptide USA.
2. Lenti-virus particles 2×10⁹ [IU/1 ml] for IM twice/three times a week administration Lentivirus particles are produced by Sirion Germany.

### Treatment Period

### Days 1-4 (Baseline Evaluations and Dose-Titration)

### Day 1.

The following safety assessments and baseline evaluations are performed: Demographics (age, weight, height); concomitant medication; vital signs (breathing rate, heart rate, temperature, blood pressure); full physical examination including the following systems: skin, lymph nodes, head, eye, ear, nose and throat, respiratory, cardiovascular, gastrointestinal, neurologic and muscoskeletal; and blood tests (biochemistry: complete panel including renal function, liver function and CRP; hematology—White Blood Cells (WBC), hemoglobin, neutrophils, and platelets; coagulation—Prothrombin Time (PT), activated Partial Thromboplastin Time (PTT) and International Normalized Ratio (INR); inflammatory markers assessment; tumor specific biomarkers (such as CA19-9); and determination of membranal CD24 marker from tumor biopsy).
The physician assesses eligibility and determine whether the patient can continue to receive first titration dose.

### Dose Titration

A. SC administration of INS HCl peptide in the formulation of the present invention (20 mg) half an hour pre-LV administration.
B. Anti-CD24-LV full-dose administration (LM, 2×10⁹ TU/ml)
C. SC administration of INS peptide (20 mg) 3 hours post-LV administration.
D. SC administration of INS peptide (20 mg) 3 hours post last INS administration.
E. Next Day—SC administration of INS peptide (20 mg) 20-24 hours post-LV administration.
In cases there are issues with AEs/SAEs that according to the physician decision justifies reduction of the daily dose, the latest tolerable dose is administered until the end of the treatment period.

### Days 5-30

The following assessments are performed:
AE/SAE assessments
Vital signs (breathing rate, heart rate, temperature, blood pressure)
Full physical examination including skin, lymph nodes, head, eye, ear, nose and throat, respiratory, cardiovascular, gastrointestinal, neurologic and muscoskeletal
Concomitant medication
Blood tests (blood tests are taken at the baseline, after 15 days and after 30 days of treatment as part of end of treatment assessments). Biochemistry: complete panel including renal function, liver function and CRP; Hematology—White Blood Cells (WBC), hemoglobin, neutrophils, and platelets; Coagulation—Prothrombin Time (PT), activated Partial Thromboplastin Time (PTT) and International Normalized Ratio (INR); Inflammatory markers assessment; and Tumor specific biomarkers (such as CA19-9)
   Day 30 (End of Treatment Assessments) Final treatment assessments are performed after last treatment dose with Anti-CD24-LV and INS peptide. The following assessments are be performed: AE/SAE assessments; Vital signs (breathing rate, heart rate, temperature, blood pressure); Full physical examination including skin, lymph nodes, head, eye, ear, nose and throat, respiratory, cardiovascular, gastrointestinal, neurologic and muscoskeletal; Concomitant medication, Blood tests as described for days 5-30; Objective tumor response via using CT or MRI (as soon as possible after end of treatment assessments) and Evaluation of progression free survival.

### Follow-up

If improvement is seen, the treatment is continued, otherwise, stopped. If needed, the patient continues to receive the regular second-line chemotherapy gemcitabine plus NAB paclitaxel and other standard of care therapies as per hospitals standards. objective tumor response and progression free survival is followed.

### Treatment Criteria:

### Inclusion:

1. Male or female patients aged >18 years.
2. Cancer patient proven either by histology (surgical biopsy) or cytology (CT- or endoscopic-guided), with positive to membranal CD24.
3. Patients with measurable or evaluable disease according to the response evaluation criteria in solid tumors
4. Patients eligible for second-line therapy after failing first-line therapy.
5. Recovered from reversible toxicities of prior therapy
6. Patients with a malignancy that is currently not amenable to surgical intervention, due to either medical contraindications or non-resectability of the tumor.
7. ECOG performance status 0-1 with anticipated life expectancy of >3 months.
8. Adequate baseline organ function (hematologic, liver, renal and nutritional)
9. Patients, both male and female, who are either not of childbearing potential or who agree to use a medically effective method of contraception during the study and for 3 months after the last dose of treatment drug.
10. Patients with the ability to understand and give written informed consent for receiving this treatment, including all evaluations and procedures as specified by this protocol.

### Exclusion:

1. Active infection or other serious illness or autoimmune disease
2. Treatment with live attenuated vaccines in the last three weeks
3. Viral syndrome diagnosed during the two weeks before inclusion
4. Women who are pregnant or lactating. Women of child-bearing potential (WOCBP) and fertile men with a WOCBP partner, not using adequate birth control.
5. Patients with any hematologic abnormalities at baseline.
6. Patients with any serum chemistry abnormalities at baseline:
7. Patients with a significant cardiovascular disease or condition.
8. Patients with a history of human immunodeficiency virus (HIV) or active infection with hepatitis B virus (HBV) or hepatitis C virus (HCV).
9. Patients with inadequate recovery from any prior surgical procedure, or patients having undergone any major surgical procedure within 1 month prior to first study drug administration.
10. Patients with any other life-threatening illness, significant organ system dysfunction, or clinically significant laboratory abnormality, which, in the opinion of the Investigator, would either compromise the patient's safety or interfere with evaluation of the safety of the study drug
11. Patients with a psychiatric disorder or altered mental status that would preclude understanding of the informed consent process and/or completion of the necessary studies
12. Patients with the inability, in the opinion of the Investigator, to comply with the protocol requirements
13. Females who are pregnant or breast-feeding
14. Participation in an investigational drug or device study within 14 days of the first day of dosing on this study
Although the present invention has been described herein above by way of preferred embodiments thereof, it can be modified, without departing from the spirit and nature of the subject invention as defined in the appended claims.

1. A pharmaceutical composition comprising
between about 0.1 to about 30 mg/ml of a salt of a peptide comprising amino acid sequence SEQ ID NO: 1 (INS);
between about 0.1 wt % to about 15 wt % of a poloxamer having the structure of Formula I; 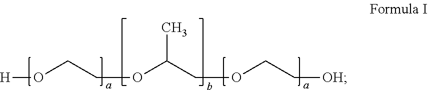
and
a pharmaceutically acceptable carrier, wherein the pH of the composition is between about 4 to about 7.5, and wherein a is an integer from 50 to 120, and b is an integer from 15 to 40.

2. The pharmaceutical composition of claim 1, wherein the salt of INS is a hydrochloride salt (INS HCl).

3. The pharmaceutical composition of claim 1, wherein a is an integer from 60 to 90 and b is an integer from 25 to 35.

4. The pharmaceutical composition of claim 3, wherein a=80 and b=21 (poloxamer 188).

5. The pharmaceutical composition of claim 4, wherein the composition comprises from about 1 wt % to about 10 wt % or from about 0.5 wt % to about 5 wt % of poloxamer 188.

6. The pharmaceutical composition of claim 5, wherein the composition comprises from about 1 mg/ml to about 20 mg/ml of the peptide INS as HCl salt.

7. The pharmaceutical composition of claim 1, comprising from about 1 to about 10 wt % of poloxamer 188 and from about 5 mg/ml to about 15 mg/ml of the peptide INS as HCl salt.

8. The pharmaceutical composition of claim 5, wherein the composition comprises: from about 0.5 wt % to about 3 wt % of poloxamer 188 and from 2 to 12 mg/ml of the peptide salt INS HCl.

9. The pharmaceutical composition of claim 1, further comprising a saccharide selected from a disaccharide and polysaccharide.

10. The pharmaceutical composition of claim 9, comprising from about 1 wt % to about 20 wt % of the saccharide.

11. (canceled)

12. The pharmaceutical composition of claim 11, wherein the disaccharide is lactose.

13. (canceled)

14. The pharmaceutical composition of claim 1, comprising from about 8 to 12 wt % lactose and:
(i) from about 0.5 to about 2 wt % of poloxamer 188, and from about 1 mg/ml to about 5 mg/ml of INS HCl; or
(ii) from about 0.8 to about 2 wt % of poloxamer 188, and from about 5 mg/ml to about 10 mg/ml of INS HCl; or
(iii) from about 0.8 to about 2 wt % of poloxamer 188, and from about 1 mg/ml to about 15 mg/ml of INS HC 1; or
(iv) from about 0.5 to about 2 wt % of poloxamer 188, and from about 1 mg/ml to about 12 mg/ml of INS HCl.

15-23. (canceled)

24. A solid pharmaceutical composition comprising a salt of a peptide comprising amino acid sequence SEQ ID NO: 1 and a poloxamer having a structure of Formula I, 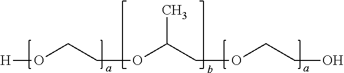
Formula I, wherein a is an integer from 50 to 120, and b is an integer from 15 to 40 and wherein the weight ratio between said peptide salt and said poloxamer is from about 10:5 to about 1: 1500.

25-34. (canceled)

35. A hydrogen halide salt of a peptide comprising SEQ ID NO: 1 or SEQ ID NO: 2, wherein said peptide comprises about 15 to 30 amino acids.

36-40. (canceled)

41. A pharmaceutical combination comprising the pharmaceutical-composition of claim 1 and one or more of the following:
(i) a linear molecule of double-stranded DNA (dsDNA) comprising long term repeat (LTR) sequences recognized by the integrating enzyme of (ii);
(ii) an integrating enzyme, capable of entering the nuclei of cells after binding to the LTR sequences of the dsDNA molecule of (i) and creating multiple double strand breaks (DSBs) in the chromosomal DNA of the cells; and optionally (iii) a targeting moiety.

42. A method of treating a disease selected from viral infection and cancer, in a subject in need thereof, the method comprising administering to the subject a pharmaceutical composition of claim 1.

43. The method according to claim 42 wherein the viral infection disease is HIV-1 infection or AIDS.

44. The method of claim 43, comprising co-administration of the peptide INS HCl with at least one additional anti-viral agent.

45. The method of claim 44, wherein the at least one additional anti-viral agent is a protease inhibitor.

46. The method of claim 45, comprising daily administration of lopinavir 400 to 1000 mg and ritonavir 100 to 300 mg.

47. The method according to claim 42, wherein the disease is cancer and the method comprises co-administration of lentiviral particles containing an antibody directed against CD24.

48-50. (canceled)


SEQUENCE LISTING (2 sequences)

SEQ ID NO: 1 - protein, 16 aa, Artificial Sequence
  Description of Artificial Sequence Synthetic peptide
WTAVQMAVFIHNFKRK

SEQ ID NO: 2 - protein, 15 aa, Artificial Sequence
  Description of Artificial Sequence Synthetic peptide
TAVQMAVFIHNFKRK